(19) United States
(12) Patent Application Publication
(10) Pub. No.: US 2021/0379077 A1
(43) Pub. Date: Dec. 9, 2021

(54) TOPOISOMERASE II CATALYTIC INHIBITOR COMPOUND THERAPEUTICS FOR CANCER TREATMENT, METHODS AND USES

(71) Applicant: The University of British Columbia, Vancouver, British Columbia (CA)

(72) Inventors: Artem Tcherkassov, Vancouver (CA); Zaccary Alperstein, Vancouver (CA); Xuesen Dong, Vancouver (CA); Ning Xie, Vancouver (CA)

(21) Appl. No.: 17/286,103

(22) PCT Filed: Aug. 30, 2019
(86) PCT No.: PCT/CA2019/051209

§ 371 Date: Apr. 16, 2021

Related U.S. Application Data
(60) Provisional application No. 62/747,821, filed on Oct. 19, 2018.

(51) Int. Cl. A61K 31/55; A61K 31/4439; A61K 31/167; A61K 31/496; A61K 31/4365; A61K 45/06

(52) U.S. Cl. CPC A61K 31/55; A61K 31/4439; A61K 45/06; A61K 31/496; A61K 31/4365; A61K 31/167

(57) ABSTRACT

II and III Provided herein are Topoisomerase II inhibitory compounds having the structure of Formulas II and III and compositions thereof for use in the treatment of cancer. In particular, the Topoisomerase II inhibitory compounds described herein may be used as catalytic inhibitors of Topoisomerase II and used for the treatment of cancer.

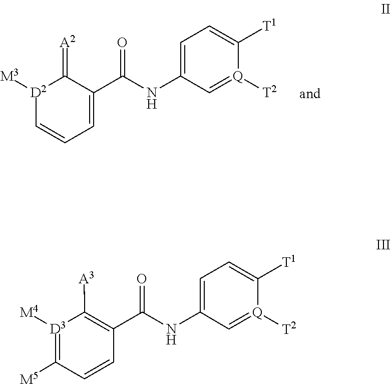

### CROSS REFERENCE TO RELATED APPLICATION

This application claims the benefit of U.S. Provisional Patent Application Ser. No. 62/747,821 filed on 19 Oct. 2018, entitled “TOPOISOMERASE II CATALYTIC INHIBITOR COMPOUND THERAPEUTICS FOR CANCER TREATMENT, METHODS AND USES ASSOCIATED THEREWITH”.

### TECHNICAL FIELD

The present invention relates to the field of Topoisomerase II inhibitors. In particular, the invention relates to Topoisomerase II inhibitor compounds for use in the treatment of cancer.

### BACKGROUND

During cell division, DNA has to replicate rapidly at an average speed of 2 kilobases per minute, this imposes strict requirements on DNA topology where supercoiled secondary structures are highly unfavorable¹. Topoisomerases paly an essential role in relieving supercoiling in DNA for its further manipulation. In particular, human topoisomerase IIα removes (i.e., relaxes) positive DNA supercoils >10-fold faster than negative supercoils and topoisomerase IIα, is involved in DNA replication. In contrast, topoisomerase IIβ, which is not believed to play a role in DNA replication, relaxes positive and negative super-helical twists at similar rates²⁰.

Topoisomerase II (Topo II) is a well-known cancer therapeutic target and Topoisomerase inhibitors represent some of the most significant chemotherapeutic drugs currently used for the treatment of human malignancies. For example, Etoposide was introduced into the clinic in 1971 and remains one of the most used chemotherapeutic compounds for a large variety of solid and hematological tumors. However, Etoposide's toxicity makes effective at targeting rapidly dividing cells, but also has undesirable off-target activities. Etoposide is a member of the class of topoisomerase inhibitors known as poisons. Poisons stabilize double stranded-DNA (dsDNA) breaks by binding to the Topo II-DNA complex, leading to cell death. Unfortunately, poisons are also associated with harsh off-target effects, and in some cases even secondary leukemia². Another class of Topo II inhibitors are known as catalytic inhibitors. Catalytic inhibitors do not stabilize dsDNA breaks and are therefore highly valued. Catalytic Topoisomerase II inhibitors target the N-terminal ATPase domain of Topoisomerase II and prevent Topoisomerase II-mediated DNA cleavage without stabilizing DNA-topo II-cleavable complexes. A number of catalytic Topoisomerase II inhibitors are known (i.e. Dexrazoxane (ICRF), Sobuzoxane (MST-16) and Merbarone). Unfortunately, and thus far, the catalytic inhibitors tested have had poor translation to the clinic³.

### SUMMARY

The present invention is based in part, on the surprising discovery that the compounds described herein modulate Topoisomerase II catalytic activity. Specifically, some compounds identified herein, also show inhibition of Topoisomerase II catalytic activity in cancer cells. For example, cancer cells may be selected from one or more of the following: cervical cancer; small-cell lung cancer; testicular cancer; carcinoma; neuroblastoma; osteosarcoma; glioblastoma; melanoma; lymphoma; leukemia; esophageal cancer; stomach cancer; colon cancer; breast cancer; ovarian cancer; endometrial cancer; chondrosarcomas; central nervous system cancer; liver cancer; and prostate cancer.

In accordance with one embodiment, there is provided a method of inhibiting Topoisomerase II, the method including administering a compound having the structure of Formula II:

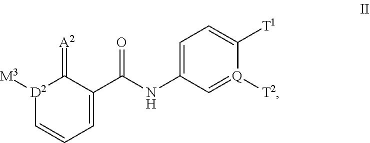

wherein, A² may be selected from S and O; D² may be selected from N and CH; M³ may be H; Q may be selected from C and N; T¹ may be selected from H,

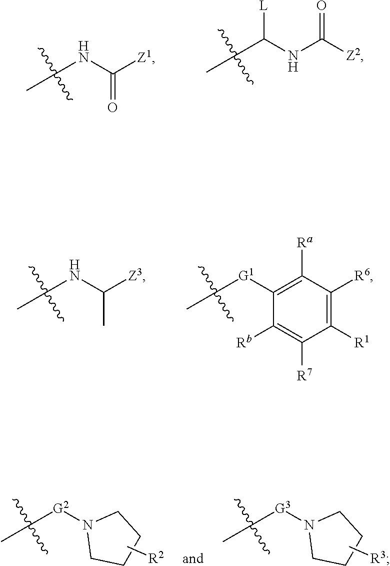

T² may be absent when Q is N or may be selected from H, F, Cl, Br,

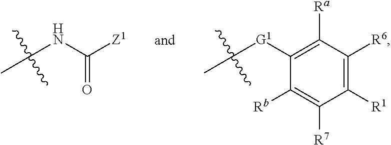

when Q may be C, provided that both T¹ and T² are not both H; Z¹ may be selected from H, CH₂CH₃, CH₂CH₂CH₃, CH(CH₃)CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, OC(CH₃) (CH₃)CH₃,

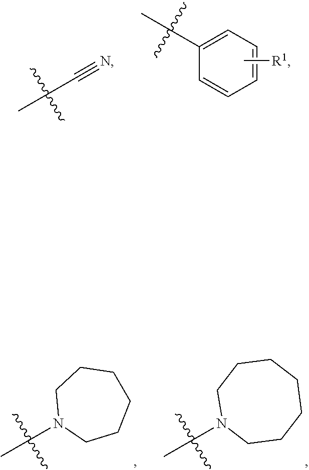

F, Cl, Br and CF₃; Z² may be selected from H, CH₃, CH₂CH₃, CH₂CH₂CH₃, CH(CH₃)CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, OC(CH₃) (CH₃)CH₃,

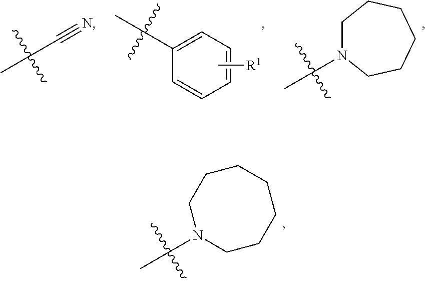

F, Cl, Br and CF₃; Z³ may be selected from H, CH₃, CH₂CH₃, CH₂CH₂CH₃, CH(CH₃)CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, OC(CH₃) (CH₃)CH₃,

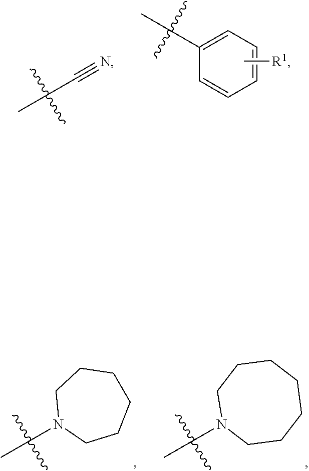

F, Cl, Br and CF₃; L may be selected from H, CH₃, CH₂CH₃, CH(CH₃)CH₃, NH₂, OH, OCH₃, OCH₂CH₃, F, Cl, Br and CF₃; G¹ may be selected from O, S, CH₂ and NH; G² may be selected from O, S, CH₂ and NH; G³ may be selected from O, S, CH₂ and NH; R^{a} and R^{b} may be H; R¹ may be independently selected from H, CH₃, CH₂CH₃, CH₂CH₂CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, NHC(═O)CH₂CH₃,

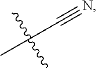 [SMILES: *C#N]

F, Cl, Br and CF₃; R² may be independently selected from H, CH₃, CH₂CH₃, CH₂CH₂CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH_{3J} NHC(═O)CH₂CH₃,

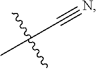 [SMILES: *C#N]

F, Cl, Br and CF₃; R³ may be independently selected from H, CH₃, CH₂CH₃, CH₂CH₂CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, NHC(═O)CH₂CH₃,

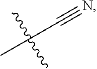 [SMILES: *C#N]

F, Cl, Br and CF₃; R⁶ may be selected from H, CH₃, CH₂CH₃, CH₂CH₂CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, NHC(═O)CH₂CH₃,

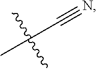 [SMILES: *C#N]

F, Cl, Br and CF₃; and R⁷ may be selected from H, CH₃, CH₂CH₃, CH₂CH₂CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, NHC(═O)CH₂CH₃,

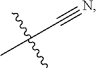 [SMILES: *C#N]

### F, Cl, Br and CF₃.

In accordance with another embodiment, there is provided a method of inhibiting Topoisomerase II, the method including administering a compound having the structure of Formula III:

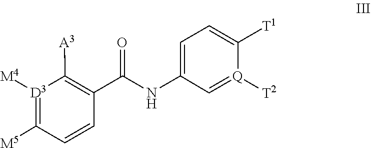

wherein, A³ may be selected from SH, OH and OCH₃; D³ may be selected from N and C; alternatively, A³ may be H or SCH₃ when D³ is C; M³ may be absent or is selected from H, Cl, F, Br; M³ may be selected from H, CH₃, Cl, F and Br; Q may be selected from C and N; T¹ may be selected from H,

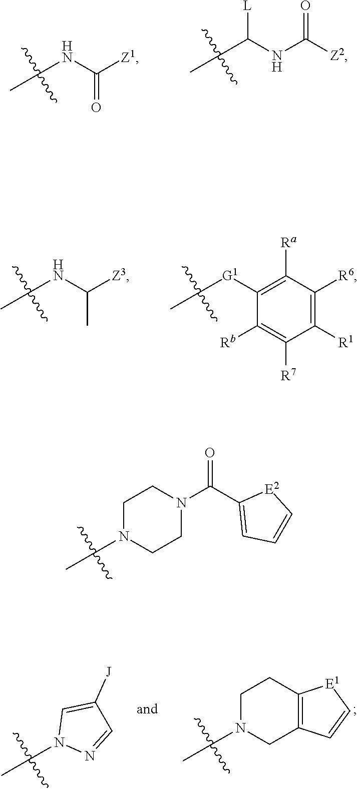

T² may be absent when Q is N or may be selected from H, F, Cl, Br,

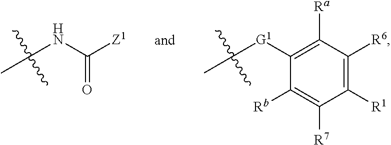

when Q is C, provided that both T¹ and T² are not both H; Z¹ may be selected from H, CH₂CH₃, CH₂CH₂CH₃, CH(CH₃)CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, OC(CH₃) (CH₃)CH₃,

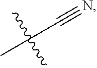 [SMILES: *C#N]

F, Cl, Br and CF₃; Z² may be selected from H, CH₃, CH₂CH₃, CH₂CH₂CH₃, CH(CH₃)CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, OC(CH₃) (CH₃)CH₃,

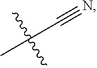 [SMILES: *C#N]

F, Cl, Br and CF₃; Z³ may be selected from H, CH₃, CH₂CH₃, CH₂CH₂CH₃, CH(CH₃)CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, OC(CH₃) (CH₃)CH₃,

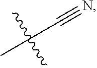 [SMILES: *C#N]

F, Cl, Br and CF₃; L may be selected from H, CH₃, CH₂CH₃, CH(CH₃)CH₃, NH₂, OH, OCH₃, OCH₂CH₃, F, Cl, Br and CF₃; G¹ may be selected from O, S, CH₂ and NH; R^{a} and R^{b} may be H; R¹ may be selected from OCH₃ and NHC(═O)CH₂CH₃; R⁶ may be selected from H, CH₂CH₂CH₃, CH₂CH(CH₃)CH₃, CH(CH₃)CH₃, OCH₂CH₃, OCH₂CH₂CH₃, OCH₂CH(CH₃)CH₃, OCH(CH₃)CH₃, NHC(═O)CH₂CH₃,

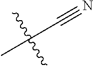 [SMILES: *C#N]

and CF₃; R⁷ may be selected from H, CH₂CH₂CH₃, CH₂CH(CH₃)CH₃, CH(CH₃)CH₃, OCH₂CH₃, OCH₂CH₂CH₃, OCH₂CH(CH₃)CH₃, OCH(CH₃)CH₃, NHC(═O)CH₂CH₃,

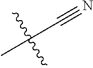 [SMILES: *C#N]

and CF₃; J may be selected from H, CH₃, NH₂, OH, OCH₃, F, Cl, Br and CF₃; E¹ may be selected from O, S, CH₂ and NH; and E² may be selected from O, S, CH₂ and NH.

The inhibiting of Topoisomerase II may be catalytic inhibition. The inhibiting of Topoisomerase II may be for the treatment of cancer. The cancer may be selected from one or more of the following: cervical cancer; small-cell lung cancer; testicular cancer; carcinoma; neuroblastoma; osteosarcoma; glioblastoma; melanoma; lymphoma; leukemia; esophageal cancer; stomach cancer; colon cancer; breast cancer; ovarian cancer; endometrial cancer; chondrosarcomas; central nervous system cancer; liver cancer; and prostate cancer. The prostate cancer may be selected from: Neuroendocrine Prostate Cancer (NEPC); Prostate Adenocarcinoma; castration resistant prostate cancer (CRPC); androgen receptor pathway inhibitor (ARPI) resistant prostate cancer; enzalutamide (ENZ)-resistant (ENZ^{R}); and Abiraterone (Abi)-resistant (ABI_{R}). The compound may be selected from one or more of the following:

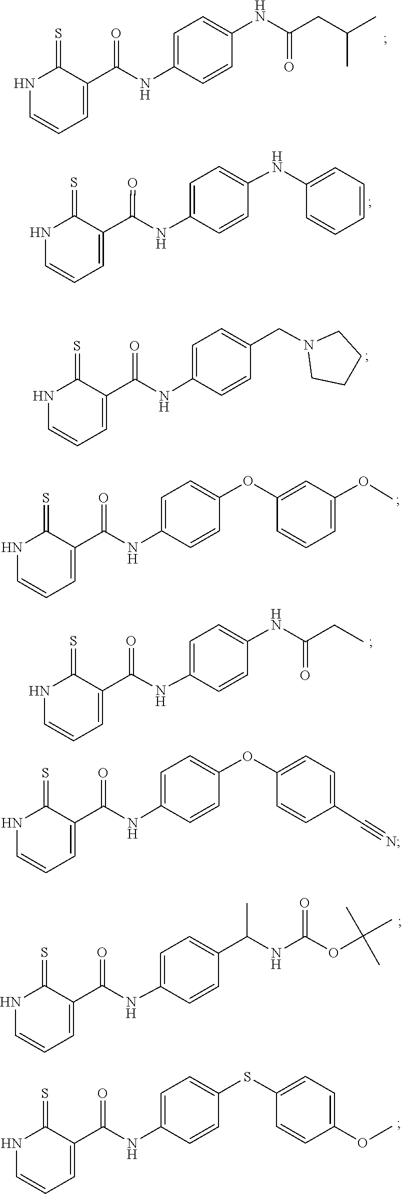

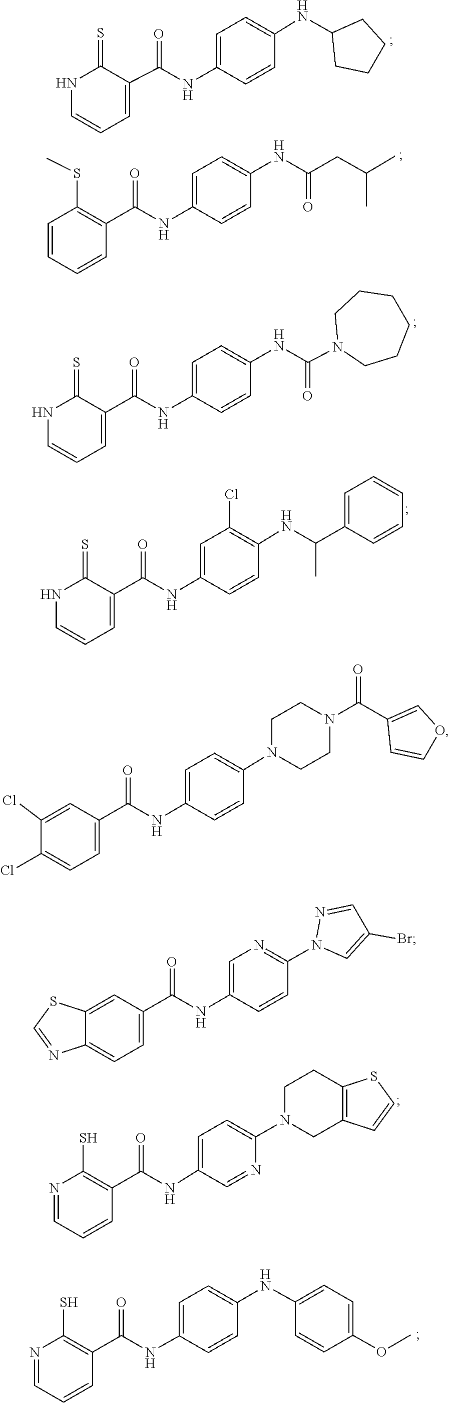

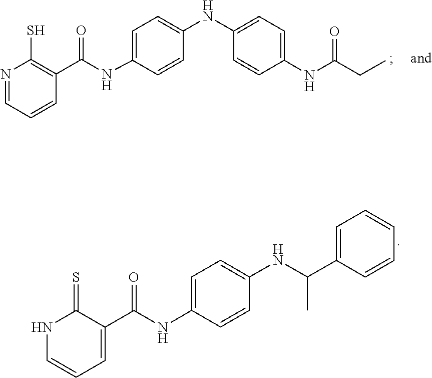 [SMILES: CCC(=O)Nc1ccc(Nc2ccc(NC(=O)c3cccnc3S)cc2)cc1 ; CC(Nc1ccc(NC(=O)c2ccc[nH]c2=S)cc1)c1ccccc1]

The compound of Formula II or III may be administered in combination with a taxol and/or a Topoisomerase poison and/or an androgen receptor (AR) therapy for the treatment of prostate cancer.

In accordance with another embodiment, there is provided a pharmaceutical composition for treating cancer, comprising compound of Formula II or III and a pharmaceutically acceptable carrier.

In accordance with another embodiment, there is provided a use of a compound of Formula II or III for treating cancer.

In accordance with another embodiment, there is provided a use of a compound of Formula II or III in the manufacture of a medicament for treating cancer.

In accordance with another embodiment, there is provided a commercial package comprising (a) a compound of any one of Formula II or III and a pharmaceutically acceptable carrier; and (b) instructions for the use thereof for treating cancer.

In accordance with another embodiment, there is provided a commercial package comprising (a) a pharmaceutical composition comprising a compound of any one of Formula II or III and a pharmaceutically acceptable carrier; and (b) instructions for the use thereof for treating cancer.

In accordance with another embodiment, there is provided a method of inhibiting Topoisomerase II, the method comprising administering a compound having the structure

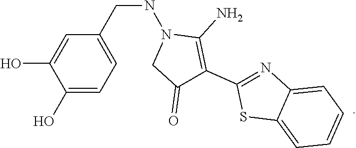 [SMILES: NC1=C(c2nc3ccccc3s2)C(=O)CN1NCc1ccc(O)c(O)c1]

In accordance with another embodiment, there is provided a compound for use in the treatment of cancer, the compound may be selected from one or more of the following:

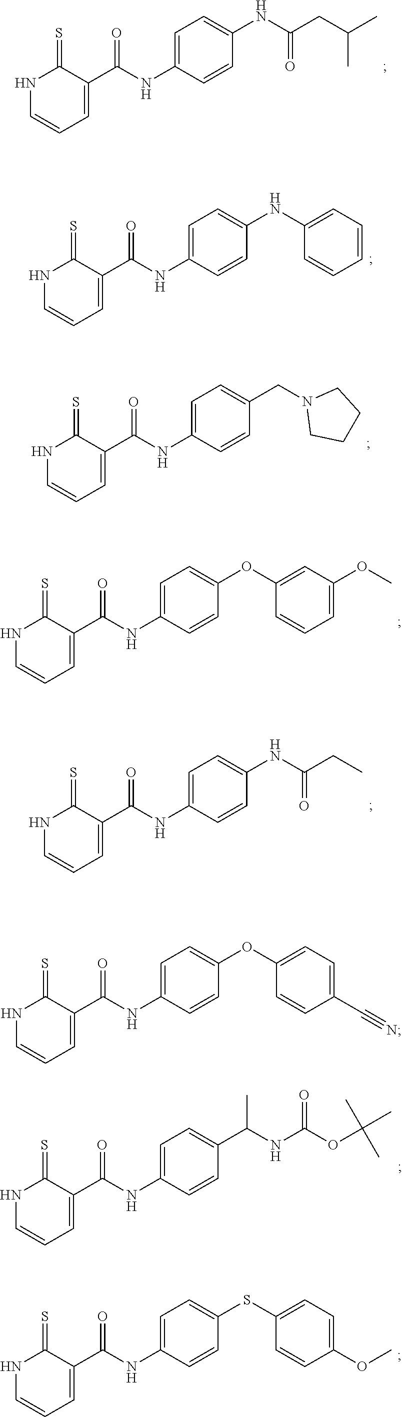

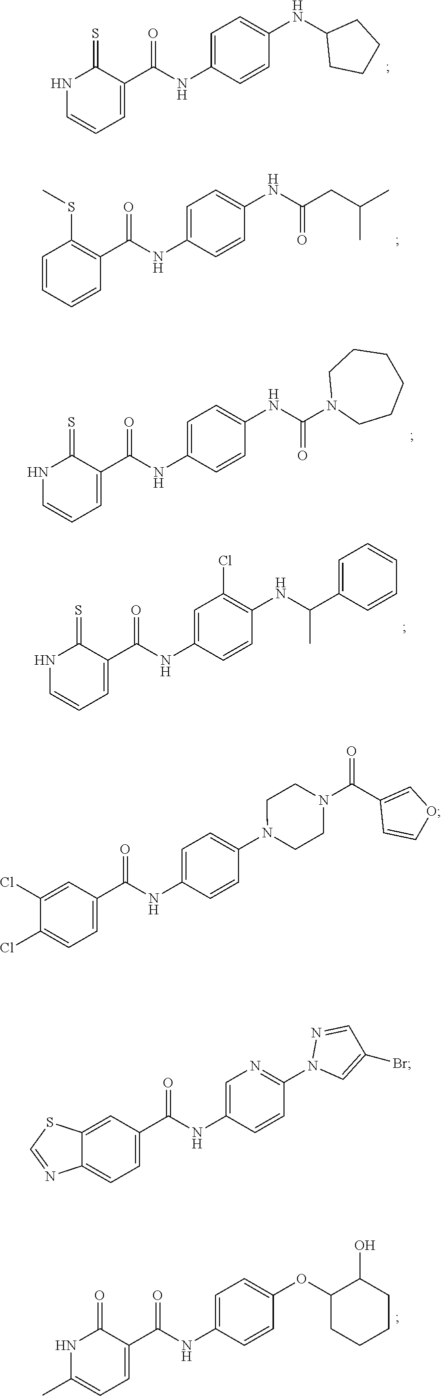

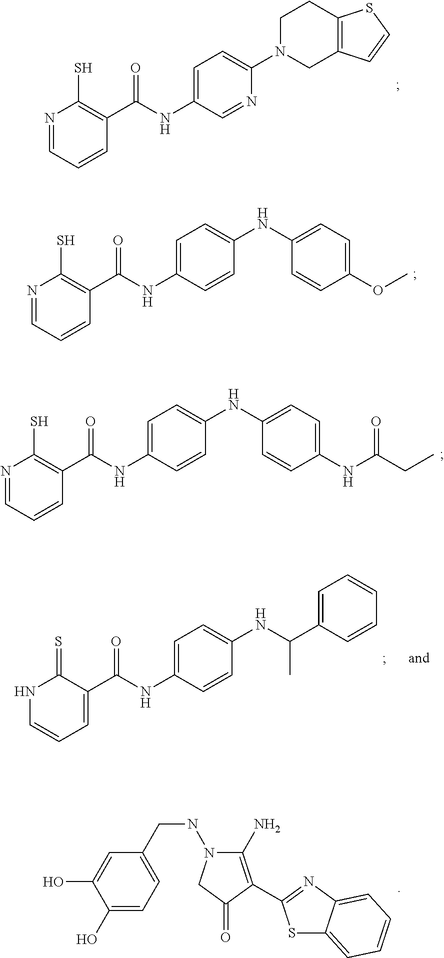 [SMILES: O=C(Nc1ccc(N2CCc3sccc3C2)nc1)c1cccnc1S ; COc1ccc(Nc2ccc(NC(=O)c3cccnc3S)cc2)cc1 ; CCC(=O)Nc1ccc(Nc2ccc(NC(=O)c3cccnc3S)cc2)cc1 ; CC(Nc1ccc(NC(=O)c2ccc[nH]c2=S)cc1)c1ccccc1 ; NC1=C(c2nc3ccccc3s2)C(=O)CN1NCc1ccc(O)c(O)c1]

The treatment of cancer may be by inhibition of Topoisomerase II. The treatment of cancer may be by catalytic inhibition of Topoisomerase II. The cancer may be selected from one or more of the following: cervical cancer; small-cell lung cancer; testicular cancer; carcinoma; neuroblastoma; osteosarcoma; glioblastoma; melanoma; lymphoma; leukemia; esophageal cancer; stomach cancer; colon cancer; breast cancer; ovarian cancer; endometrial cancer; chondrosarcomas; central nervous system cancer; liver cancer; and prostate cancer. The prostate cancer may be selected from: Neuroendocrine Prostate Cancer (NEPC); Prostate Adenocarcinoma; castration resistant prostate cancer (CRPC); androgen receptor pathway inhibitor (ARPI) resistant prostate cancer; enzalutamide (ENZ)-resistant (ENZ^{R}); and Abiraterone (Abi)-resistant (ABI_{R}). The compound may be used in combination with a taxol and/or a Topoisomerase poison and/or an androgen receptor (AR) therapy for the treatment of prostate cancer. The compound may be selected from

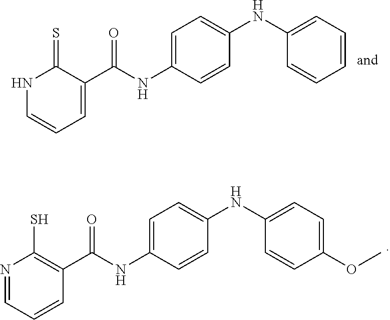 [SMILES: O=C(Nc1ccc(Nc2ccccc2)cc1)c1ccc[nH]c1=S ; COc1ccc(Nc2ccc(NC(=O)c3cccnc3S)cc2)cc1]

The compound may have the structure of Formula II or Formula III:

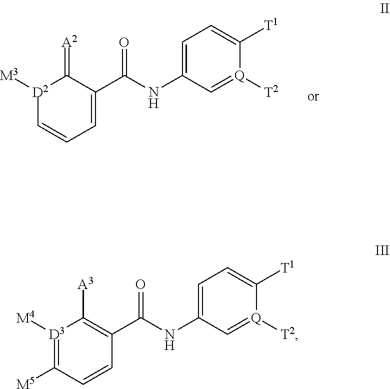

wherein, A² may be selected from S and O; A³ may be selected from SH, SCH₃, OH and OCH₃; D² may be selected from N and CH; D³ may be selected from N and C; M³ may be H; M⁴ may be absent or may be selected from H, Cl, F, Br; and M³ may be selected from H, CH₃, Cl, F and Br.

The inhibiting of Topoisomerase II may be catalytic inhibition. The inhibiting Topoisomerase II may be for the treatment of cancer. The cancer may be selected from one or more of the following: cervical cancer; small-cell lung cancer; testicular cancer; carcinoma; neuroblastoma; osteosarcoma; glioblastoma; melanoma; lymphoma; leukemia; esophageal cancer; stomach cancer; colon cancer; breast cancer; ovarian cancer; endometrial cancer; chondrosarcomas; central nervous system cancer; liver cancer; and prostate cancer. The prostate cancer may be selected from: Neuroendocrine Prostate Cancer (NEPC); Prostate Adenocarcinoma; castration resistant prostate cancer (CRPC); androgen receptor pathway inhibitor (ARPI) resistant prostate cancer; enzalutamide (ENZ)-resistant (ENZ^{R}); and Abiraterone (Abi)-resistant (ABI_{R}). The compound of Formula II or III may be administered in combination with taxols and/or Topoisomerase poisons, as well as combinations with androgen receptor (AR) therapies for the treatment of prostate cancer. The compound of Formula II or III may be administered in combination with a taxol and/or a Topoisomerase poison and/or an androgen receptor (AR) therapy for the treatment of prostate cancer.

### BRIEF DESCRIPTION OF THE DRAWINGS

FIG. 1 shows compound #60 (T60 or Top60) inhibits TOP2 activity (a) Catenated kinetoplast DNA was incubated with human TOP2a in the presence of 50 uM Etoposide, 50 uM ICRF193 or 0-50 μM of Top60 compound at 37° C. for 1 hour. DNA samples were separated by electrophoresis on 0.8% agarose gels and visualized by ethium bromide staining. The positions of K-DNA and decatenated (D) DNA products are marked by arrows. Experiments were repeated five times and the densitometry of the D bands were used to establish an inhibition curve to calculate IC50. (b) Supercoiled pHOT™ plasmid was incubated with human TOP2a in the presence of 50 μM Etopside, 50 uM ICRF193 or 0-50 μM of compound #60 (Top60) compound at 37° C. for 1 hour. DNA products were separated by electrophoresis on 1% agarose gels and visualized by ethium bromide staining. The positions of supercoiled (SC), nicked (N), and relaxed (R) DNA bands are marked by arrows. Experiments were repeated five times and the densitometiy of the SC bands were used to establish an inhibition curve to calculate IC50. (c) Supercoiled pHOT™ plasmid was incubated with Topoisomerase I prior to addition of either mAMSA or compound #60 (Top60) at concentrations of 0, 5, 10, 25 and 50 μM at 37° C. for 30 minutes. Reactions were stopped by 1% SDS at 37° C. for 15 minutes. DNA products were separated by electrophoresis on a 1% agarose gels and visualized by ethium bromide staining. The positions of supercoiled (SC) and relaxed (R) DNA bands are marked by arrows, (d) Cleavage assay: Supercoiled pHOT plasmid was used to perform DNA cleavage assays in the presence of vehicle, 50 μM of Etopoisde, ICRF193 and compound #60 (Top60), respectively. The positions of supercoiled (SC), nicked (N), and relaxed (R) DNA bands are marked by arrows.

FIG. 2 shows Top60 inhibits DNA synthesis at S phase during cell cycling (a) Hela cells were treated with vehicle or 20 μM of Top60, Etoposide or ICRF187 for 48 hours. BrdU incorporation rates were determined by Millipore™ BrdU incorporation kit (CAT #2750). (b-c) Hela cells were treated with vehicle or 20 μM of Top60 for 48 hours. Cells were then stained with APC-BrdU and 7-AAD for 4 hours, and subjected to FACS assays to determine cell populations at Sub G0/G1, G0/G1, S, and G2/M phases. Results were collected from three independent experiments, (d-e) Hela cells were treated with either vehicle or 20 μM T60 for 48 hours. Cells were fixed and co-stained with DAPI and phalloidin, and examined by confocal microscopy. Fifty cells were chosen from five high power fields to analyze nuclei sizes by the Image J software. Representative bright field microscopic images were also shown. Scale bar=50 μm.

FIG. 3 shows Top60 inhibits cell proliferation, but has low cytotoxicity, wherein (a) Hela cells were treated with 0, 1, 5, 10, 15 and 20 μM of Top60, Etoposide and ICRF187 for 48 hours. Cytotoxicity was evaluated by measuring LDH levels from cell culture media; (b) K562 cells were treated with either vehicle or 10-20 μM of Top60, Etoposide and ICRF187 for 4 hours. Whole cell lyses were collected to measure γH2AX levels with vinculin as the loading control; (c) K562 cells were treated with 0, 1, 5, 10, 15 and 20 μM of Top60, 20 μM Etoposide or 20 uM ICRF187 for 48 hours. Cell proliferation rates were determined by MTS assays; and (d) K562 cells were treated with 20 μM of Top60, Etoposide or ICRF187 for 0, 24, 48 and 72 hours. Cell proliferation rates were measured by MTS assays. All results were collected from three independent experiments.

FIG. 4 shows on-target effects of Top60 in cells, wherein (a) K562 cells were treated with vehicle or 20 μM Top60 for 2 hours, before exposing different temperature points as indicated. Whole cell lyses were collected and used to measure protein levels of TOP2α, TOP2β with β-Actin as a loading control; (b) K562 cells were treated with 0, 1, and 10 μM of Top60 for 24 hours. Protein lyses were collected from soluble nuclear extract, chromatin fraction or whole cell lysate. TOP2α and TOP2β protein levels were determined by immunoblotting with Lamin A/C, Histone H3 and β-Actin as loading controls; and (c) Etoposide-resistant K562 cell line was established by treating cells with 0.5 μM of Etoposide for 6 months. TOP2α, TOP2β, and β-actin protein levels were determined by immunoblotting. Etoposide-resistant K562 cells were treated with 0, 1, 10, 15 and 20 μM of Top60, Etoposide or ICRF187 for 48 hours. Cell proliferation rates were determined by MTS assays.

FIG. 5 shows an interaction count based histogram, wherein counts are marked at every time point for each residue (below) and the total number of interactions are in a histogram above. Note the mutual exclusivity between GLN 789 and GLY 793.

FIG. 6 shows protein-ligand interaction histogram of specific interaction types.

FIG. 7 shows a protein-ligand interaction diagram, wherein only interactions that occur more than 20% of the simulation are shown.

### DETAILED DESCRIPTION

The following detailed description will be better understood when read in conjunction with the appended figures. For the purpose of illustrating the invention, the figures demonstrate embodiments of the present invention. However, the invention is not limited to the precise arrangements, examples, and instrumentalities shown.

Despite the poor performance of previous catalytic Topoisomerase II inhibitors, it is believed by the inventors, that these previous failures in designing catalytic inhibitors do not come from their mechanism of inhibition, but from the presence of toxic scaffolds, or sub-optimal ligand binding sites. For example, ICRF (i.e. IUPAC name 4-[2-(3,5-Dioxo-1-piperazinyl)-1-methylpropyl]piperazine-2,6-dione; CAS number 21416-68-2; Dexrazoxane; ICRF-187 or ICRF-193) is a potent catalytic inhibitor of Topo II. However, the pocket in which it binds is small, limiting its scope for medicinal chemistry. A computer aided drug discovery (CADD) campaign was initiated to screen ˜6 million molecules from the ZINC 15⁴ database against a novel pocket on Topo II. This was facilitated by the implementation of consensus scoring from various virtual screening programs⁵. With our highly coordinated in-silico and wet-lab rational drug discovery pipeline we have successfully discovered and characterized a number of highly potent catalytic inhibitors. One compound in particular, compound 60, has been shown to inhibit Topo II with nanomolar IC50. Furthermore, it is demonstrated herein that the identified drug candidate does not act as a poison, as no linear DNA is formed upon incubation with Topoisomerase II in relaxation assays. Interestingly, it was found that the compound 60 likely blocks DNA replication at the decatenation checkpoint, causing the nuclei to enlarge. Finally, a mechanism of action for the lead compound is herein proposed, based on biological and in-silico experiments.

Interestingly, known Topoisomerase II inhibitor Merbarone (i.e. CAS Number 97534-21-9; 5-(N-Phenylcarbamoyl)-2-thiobarbituric acid) has structural similarities to some of the Topoisomerase II catalytic inhibitor compounds identified herein. The structure of Merbarone is

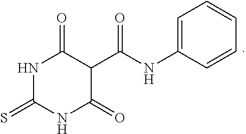 [SMILES: O=C1NC(=S)NC(=O)C1C(=O)Nc1ccccc1]

Any terms not directly defined herein shall be understood to have the meanings commonly associated with them as understood within the art of the invention.

The Topoisomerase II complex is an attractive target for direct inhibition. In silico computational drug discovery methods were used to conduct a virtual screen of more than 6 million purchasable compounds from the ZINC database (Irwin, J. et al. Abstracts of Papers Am. Chem. Soc. (2005) 230:U1009) to identify potential Topoisomerase II complex binders. The in silico methods included large-scale docking, in-site rescoring and consensus voting procedures.

It will be understood by a person of skill that COOH and NR2 may include the corresponding ions, for example carboxylate ions and ammonium ions, respectively. Alternatively, where the ions are shown, a person of skill in the art will appreciate that the counter ion may also be present.

Those skilled in the art will appreciate that the point of covalent attachment of the moiety to the compounds as described herein may be, for example, and without limitation, cleaved under specified conditions. Specified conditions may include, for example, and without limitation, in vivo enzymatic or non-enzymatic means. Cleavage of the moiety may occur, for example, and without limitation, spontaneously, or it may be catalyzed, induced by another agent, or a change in a physical parameter or environmental parameter, for example, an enzyme, light, acid, temperature or pH. The moiety may be, for example, and without limitation, a protecting group that acts to mask a functional group, a group that acts as a substrate for one or more active or passive transport mechanisms, or a group that acts to impart or enhance a property of the compound, for example, solubility, bioavailability or localization.

In some embodiments, compounds of Formulas II and III, as described herein, may be used for systemic treatment of at least one indication selected from the group consisting of: cervical cancer; small-cell lung cancer; testicular cancer; carcinoma; neuroblastoma; osteosarcoma; glioblastoma; melanoma; lymphoma; leukemia; esophageal cancer; stomach cancer; colon cancer; breast cancer; ovarian cancer; endometrial cancer; chondrosarcomas; central nervous system cancer; liver cancer; and prostate cancer. Alternatively, the compounds of Formulas II and III may be used for systemic treatment of at least one indication selected from the group consisting of: prostate cancer; breast cancer; colon cancer; cervical cancer; small-cell lung carcinoma; neuroblastomas; osteosarcoma; glioblastoma; melanoma; and myeloid leukaemia. In some embodiments compounds of Formulas II and III may be used in the preparation of a medicament or a composition for systemic treatment of an indication described herein. In some embodiments, methods of systemically treating any of the indications described herein are also provided.

Compounds as described herein may be in the free form or in the form of a salt thereof. In some embodiment, compounds as described herein may be in the form of a pharmaceutically acceptable salt, which are known in the art (Berge S. M. et al., J. Pharm. Sci. (1977) 66(1):1-19). Pharmaceutically acceptable salt as used herein includes, for example, salts that have the desired pharmacological activity of the parent compound (salts which retain the biological effectiveness and/or properties of the parent compound and which are not biologically and/or otherwise undesirable). Compounds as described herein having one or more functional groups capable of forming a salt may be, for example, formed as a pharmaceutically acceptable salt. Compounds containing one or more basic functional groups may be capable of forming a pharmaceutically acceptable salt with, for example, a pharmaceutically acceptable organic or inorganic acid. Pharmaceutically acceptable salts may be derived from, for example, and without limitation, acetic acid, adipic acid, alginic acid, aspartic acid, ascorbic acid, benzoic acid, benzenesulfonic acid, butyric acid, cinnamic acid, citric acid, camphoric acid, camphorsulfonic acid, cyclopentanepropionic acid, diethylacetic acid, digluconic acid, dodecylsulfonic acid, ethanesulfonic acid, formic acid, fumaric acid, glucoheptanoic acid, gluconic acid, glycerophosphoric acid, glycolic acid, hemisulfonic acid, heptanoic acid, hexanoic acid, hydrochloric acid, hydrobromic acid, hydriodic acid, 2-hydroxyethanesulfonic acid, isonicotinic acid, lactic acid, malic acid, maleic acid, malonic acid, mandelic acid, methanesulfonic acid, 2-napthalenesulfonic acid, naphthalenedisulphonic acid, p-toluenesulfonic acid, nicotinic acid, nitric acid, oxalic acid, pamoic acid, pectinic acid, 3-phenylpropionic acid, phosphoric acid, picric acid, pimelic acid, pivalic acid, propionic acid, pyruvic acid, salicylic acid, succinic acid, sulfuric acid, sulfamic acid, tartaric acid, thiocyanic acid or undecanoic acid. Compounds containing one or more acidic functional groups may be capable of forming pharmaceutically acceptable salts with a pharmaceutically acceptable base, for example, and without limitation, inorganic bases based on alkaline metals or alkaline earth metals or organic bases such as primary amine compounds, secondary amine compounds, tertiary amine compounds, quaternary amine compounds, substituted amines, naturally occurring substituted amines, cyclic amines or basic ion-exchange resins. Pharmaceutically acceptable salts may be derived from, for example, and without limitation, a hydroxide, carbonate, or bicarbonate of a pharmaceutically acceptable metal cation such as ammonium, sodium, potassium, lithium, calcium, magnesium, iron, zinc, copper, manganese or aluminum, ammonia, benzathine, meglumine, methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, glucamine, methylglucamine, theobromine, purines, piperazine, piperidine, procaine, N-ethylpiperidine, theobromine, tetramethylammonium compounds, tetraethylammonium compounds, pyridine, N,N-dimethylaniline, N-methylpiperidine, morpholine, N-methylmorpholine, N-ethylmorpholine, dicyclohexylamine, dibenzylamine, N,N-dibenzylphenethylamine, 1-ephenamine, N,N′-dibenzylethylenediamine or polyamine resins. In some embodiments, compounds as described herein may contain both acidic and basic groups and may be in the form of inner salts or zwitterions, for example, and without limitation, betaines. Salts as described herein may be prepared by conventional processes known to a person skilled in the art, for example, and without limitation, by reacting the free form with an organic acid or inorganic acid or base, or by anion exchange or cation exchange from other salts. Those skilled in the art will appreciate that preparation of salts may occur in situ during isolation and purification of the compounds or preparation of salts may occur by separately reacting an isolated and purified compound.

In some embodiments, compounds and all different forms thereof (e.g. free forms, salts, polymorphs, isomeric forms) as described herein may be in the solvent addition form, for example, solvates. Solvates contain either stoichiometric or non-stoichiometric amounts of a solvent in physical association the compound or salt thereof. The solvent may be, for example, and without limitation, a pharmaceutically acceptable solvent. For example, hydrates are formed when the solvent is water or alcoholates are formed when the solvent is an alcohol.

In some embodiments, compounds and all different forms thereof (e.g. free forms, salts, solvates, isomeric forms) as described herein may include crystalline and amorphous forms, for example, polymorphs, pseudopolymorphs, conformational polymorphs, amorphous forms, or a combination thereof. Polymorphs include different crystal packing arrangements of the same elemental composition of a compound. Polymorphs usually have different X-ray diffraction patterns, infrared spectra, melting points, density, hardness, crystal shape, optical and electrical properties, stability and/or solubility. Those skilled in the art will appreciate that various factors including recrystallization solvent, rate of crystallization and storage temperature may cause a single crystal form to dominate.

In some embodiments, compounds and all different forms thereof (e.g. free forms, salts, solvates, polymorphs) as described herein include isomers such as geometrical isomers, optical isomers based on asymmetric carbon, stereoisomers, tautomers, individual enantiomers, individual diastereomers, racemates, diastereomeric mixtures and combinations thereof, and are not limited by the description of the formulas illustrated for the sake of convenience.

In some embodiments, pharmaceutical compositions as described herein may comprise a salt of such a compound, preferably a pharmaceutically or physiologically acceptable salt. Pharmaceutical preparations will typically comprise one or more carriers, excipients or diluents acceptable for the mode of administration of the preparation, be it by injection, inhalation, topical administration, lavage, or other modes suitable for the selected treatment. Suitable carriers, excipients or diluents (used interchangeably herein) are those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein microparticles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20th ed., Lippencott Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene polyoxypropylene copolymers may be used to control the release of the compounds. Other potentially useful parenteral delivery systems for modulatory compounds include ethylene vinyl acetate copolymer particles, osmotic pumps, implantable infusion systems, and liposomes. Formulations for inhalation may contain excipients, for example, lactose, or may be aqueous solutions containing, for example, polyoxyethylene 9 lauryl ether, glycocholate and deoxycholate, or may be oily solutions for administration in the form of nasal drops, or as a gel.

Compounds or pharmaceutical compositions as described herein or for use as described herein may be administered by means of a medical device or appliance such as an implant, graft, prosthesis, stent, etc. Also, implants may be devised which are intended to contain and release such compounds or compositions. An example would be an implant made of a polymeric material adapted to release the compound over a period of time.

An “effective amount” of a pharmaceutical composition as described herein includes a therapeutically effective amount or a prophylactically effective amount. A “therapeutically effective amount” refers to an amount effective, at dosages and for periods of time necessary, to achieve the desired therapeutic result, such as reduced tumor size, increased life span or increased life expectancy. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as smaller tumors, increased life span, increased life expectancy or prevention of the progression of prostate cancer to an androgen independent form. Typically, a prophylactic dose is used in subjects prior to or at an earlier stage of disease, so that a prophylactically effective amount may be less than a therapeutically effective amount.

It is to be noted that dosage values may vary with the severity of the condition to be alleviated. For any particular subject, specific dosage regimens may be adjusted over time according to the individual need and the professional judgment of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In some embodiments, compounds and all different forms thereof as described herein may be used, for example, and without limitation, in combination with other treatment methods for at least one indication selected from the group consisting of: cervical cancer; small-cell lung cancer; testicular cancer; carcinoma; neuroblastoma; osteosarcoma; glioblastoma; melanoma; lymphoma; leukemia; esophageal cancer; stomach cancer; colon cancer; breast cancer; ovarian cancer; endometrial cancer; chondrosarcomas; central nervous system cancer; liver cancer; and prostate cancer. Alternatively, the compounds described herein may be useful for the treatment of one or more of the following: cervical cancer, small-cell lung cancer, testicular cancer, lymphoma, leukemia, esophageal cancer, stomach cancer, colon cancer, breast cancer, ovarian cancer, endometrial cancer, chondrosarcomas, central nervous system cancer, liver cancer and prostate cancer. For example, compounds and all their different forms as described herein may be used as neo-adjuvant (prior), adjunctive (during), and/or adjuvant (after) therapy with surgery, radiation (brachytherapy or external beam), or other therapies (for example, HIFU). Furthermore, the compounds described herein may be administered with or combined with known chemotherapeutic treatments. For example, a compound of any one of Formulas II or III may be administered in combination with taxols and Topoisomerase poisons, as well as in combination with androgen receptor (AR) therapies (for example, ADT, ARPIs, etc.) for prostate cancer (PCa).

In general, compounds as described herein should be used without causing substantial toxicity. Toxicity of the compounds as described herein can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be appropriate to administer substantial excesses of the compositions. Some compounds as described herein may be toxic at some concentrations. Titration studies may be used to determine toxic and non-toxic concentrations. Toxicity may be evaluated by examining a particular compound's or composition's specificity across cell lines using PC3 cells as a negative control that do not express AR. Animal studies may be used to provide an indication if the compound has any effects on other tissues. Systemic therapy that targets the AR will not likely cause major problems to other tissues since anti-androgens and androgen insensitivity syndrome are not fatal.

Compounds as described herein may be administered to a subject. As used herein, a “subject” may be a human, non-human primate, rat, mouse, cow, horse, pig, sheep, goat, dog, cat, etc. The subject may be suspected of having or at risk for having a cancer, such as cervical cancer; small-cell lung cancer; testicular cancer; carcinoma; neuroblastoma; osteosarcoma; glioblastoma; melanoma; lymphoma; leukemia; esophageal cancer; stomach cancer; colon cancer; breast cancer; ovarian cancer; endometrial cancer; chondrosarcomas; central nervous system cancer; liver cancer; and prostate cancer. Diagnostic methods for various cancers, such as cervical cancer, small-cell lung cancer, testicular cancer, lymphoma, leukemia, esophageal cancer, stomach cancer, colon cancer, breast cancer, ovarian cancer, endometrial cancer, chondrosarcomas, central nervous system cancer, liver cancer and prostate cancer, are known to those of ordinary skill in the art.

All compounds specifically described herein are commercially available. Furthermore, modifications to those compounds would be possible based on the knowledge of a person of skill in the art.

Various alternative embodiments and examples are described herein. These embodiments and examples are illustrative and should not be construed as limiting the scope of the invention.

### Materials and Methods

Virtual Screening of Potential Topoisomerase II Inhibitors.

The 4fm9 PDB entry for Topoisomerase II was downloaded and prepared with the Schrodinger Maestro™ protein preparation software module. Briefly, all original hydrogens were removed, re-added, crystallographic ions and waters were also removed, and the PROPKA™ optimizer at pH 7.4 was used to calculate with amino acid protonation states. The structure was then minimized using the OPLS-3™ force field. The MOE™ site-finder algorithm, which uses virtual atom probes to search the protein surface, was used to help propose suitable pockets. The pocket cavity volumes were calculated with Pock Drug™. Virtual Screening Library Preparation: From ZINC™, a database of about 400 million compounds, ˜ 6 million Drug-like molecules were downloaded with the additional criteria that they must be in-stock. Docking Grid Calculation: The grid was centered on the aforementioned pocket and calculated with Schrodingers Grid™ generation program. Glide™ Ligand Docking: Standard precision docking was used with all the other parameters set to their default values. E-Hits Docking: The top 100,000 molecules scored and ranked by Glide™ were docked with the E-hits program. Based on a consensus from both programs, the root-mean-square deviation of atomic positions or root-mean-square deviation (RMSD) between molecules docked in each program was measured and molecules were kept if their RMSD was below 2 Å. Finally ADMET™ was used with default parameters to calculate the ADMET_Risk, Tox_Risk, and CYP_Risk descriptors, where molecules were kept if they had a score less than 6.5, 3.3 and 1 respectively. ROCS™ shape similarity: The ROCS™ shape similarity program offered by Open Eye™ was used to look for similar molecules to our lead compound. Here the Implicit Dean Mills force field was used with all other parameters as default¹⁰. Pocket Volume Calculation: The Pock Drug™ software was used to calculated the pocket volume¹¹. Amber Simulation: The protein was minimized for 200 cycles with the Newton-Rafson algorithm. The system was heated to 300K with no pressure control using the SHAKE algorithm to freeze the bonds to hydrogen. A Langevin thermostat was used for this purpose. Generalized Born implicit solvent was used with PBradii. After heating, a production run was executed with 2 femto-second time steps writing every 2500 steps. The simulation was run for 9359 steps¹². 53 compounds having a good balance of Glide™ docking score and ligand efficiency and making favorable interactions with the surrounding side chains in the pocket were purchased for subsequent experimental testing.

In vitro Topo II activity assays—K-DNA decatenation assays were performed using the human Topo II assay kit (TG1001, TopoGENE™) with Etoposide and ICRF193 as poison and catalytic inhibitors respectively. DNA relaxation assays were performed by incubating the supercoiled pHOT™ plasmid with human Topo IIα (TG2000H™, TopoGENE™) at 37 degrees for one hour. Reactions were stopped by adding 10% SDS, EDTA and proteinase K. Plasmid DNAs were separated on DNA agarose gel and stained with ethidium bromide for visualization.

DNA intercalating assays—The supercoiled pHOT™ plasmid DNA was nicked by DNA Topoisomerase I (TG1015), before incubated with Topo II in the presence of increasing dosages of mAMSA or compound #60 (Top60). After adding the stop buffer, plasmid DNAs were separated on DNA agarose gel and stained with ethidium bromide for visualization.

DNA cleavage assays—The supercoiled pHOT™ plasmid DNA were incubated with human Topo IIα in the presence of Etoposide, ICRF193 and compound #60 (Top60) for at 37 degrees for 30 minutes. Reactions were stopped by adding 10% SDS, EDTA and Proteinase K.

BrDU incorporation and cell proliferation assays—Cell proliferation rates were measured by using the CellTitre™ 96 AqueousOne™ kit (Promega™) and bromodeoxyuridine (BrdU) assay kit (Millipore™) according to the manufacturer's protocol, with minor modifications as we have previously described by Li H. et al.¹⁹

FACS assays—Cell cycling was assessed by using BrdU incorporation into S-phase DNA through using the APC BrdU flow kit (BD Pharmingen™; Franklin Lakes, N.J., USA) according to manufacturer's protocol. Briefly, 1 mM of BrdU was added to cells and incubated for 6 hours. Cells were incubated with anti-BrdU antibody and stained with 7-AAD before processing the cells for flow cytometry.

Confocal microscopy—Cells were fixed with 4% paraformaldehyde, treated in 0.25% Triton X-100 for 15 mins, incubated with F-actin conjugated to Phalloidin-iFluor 488™ (Abeam™; Cambridge, UK), and mounted with DAPI staining mount (Vector Labs™; Burlingame, Calif., USA). Cells were then imaged by confocal microscopy at 63× magnification (Zeiss LSM 780™; Carl Zeiss AG™; Oberkochen, Germany).

Cytotoxicity assay—Cytotoxicity assays were performed using the commercial kit from Pierce (CAT #8078). Briefly, culture media was collected and used to measure lactate dehydrogenase (LDH) levels by a colorimetric method following manufacture's protocol.

Thermal shift and chromatin fraction assays—In the thermal shift assays, cells were treated with vehicle or compound #60 (Top60) for 1 hour, split into equal aliquots to be heated at 37 to 46 degrees in a PCR machine. Protein lyses were collected to perform immunoblotting assays with Topo II antibodies. In the chromatin fraction assays, cells were treated with vehicle or compound #60 (Top60). Both soluble and chromatin associated proteins were fractioned following the protocol described by Wysocka J et al.¹⁸. Protein lyses were collected to perform immunoblotting assays with Topo II antibodies.

### EXAMPLES

### Example 1: In Silico Identification of Hit Compounds Targeting the Topoisomerase II Site

Identifying the pocket binding site To determine the location of an efficacious binding site on Topoisomerase II a (Topo II), the protein was visualized in MOE⁶ (PDB ID: 4FM9). After examining the Van-Der-Waals protein surface, a pocket with favorable qualities was discovered on the surface of the protein containing a convex hull volume (1671.88 Å³), 54% polar residues, 14% aromatic (F, Y, H and W) and 26 residues available for binding. This pocket also happened to be right in the middle of the DNA binding site, making for a very promising drug-able site. The pocket was validated with MOEs site-finder probe software which characterizes candidate small molecule binding pockets by calculating how well the protein can accommodate pseudo-atom probes. To the extent of our knowledge, this is the first time that this site on Topo II has ever been targeted. This pocket was assessed with a molecular dynamics simulation to determine its flexibility, as a highly flexible pocket is expected to move around a lot and should therefore have a large change in RMSD through the simulation. After a 46-nanosecond simulation, the RMSD was observed to change minimally, further validating the choice of pocket. RMSD of pocket residues from Amber simulation of Topoisomerase II a, showed that residues used where: 432-434, 350-359, 321-323, 299-303, 286-289, 273-284, 223-231, and 219-222. Each frame represents 2500 steps at 2 femto-seconds each.

First Round Screening Following binding site identification, molecular screening was performed. To create a database of molecules for docking, the ZINC-15™ library with ˜400 Million compounds was filtered down to 6 million by selecting molecules which are available for purchase and have drug-like properties (as determined by Lipinski's rule of 5). Consensus scoring with molecular docking software Glide-SP™⁷ and E-hits⁸, was used as previously described⁵. Finally, we use the ADMET™⁹ software with ADMET_Risk, TOX_Risk, and CYP_Risk scores, discarding molecules with scores of more than 6.5, 3.3, and 1 respectively as recommended by the software manual. With this ranking in hand, the top ranked 53 molecules were ordered.

Medium Throughput Screening Assays A supercoiled plasmid relaxation assay was used as a first, course filter to determine candidate small molecules. Small molecule candidates that were sufficiently active in the first round screening were then subject to the kDNA decatenation assay. Out of those tested, compounds 19 and 23 were found to be most active.

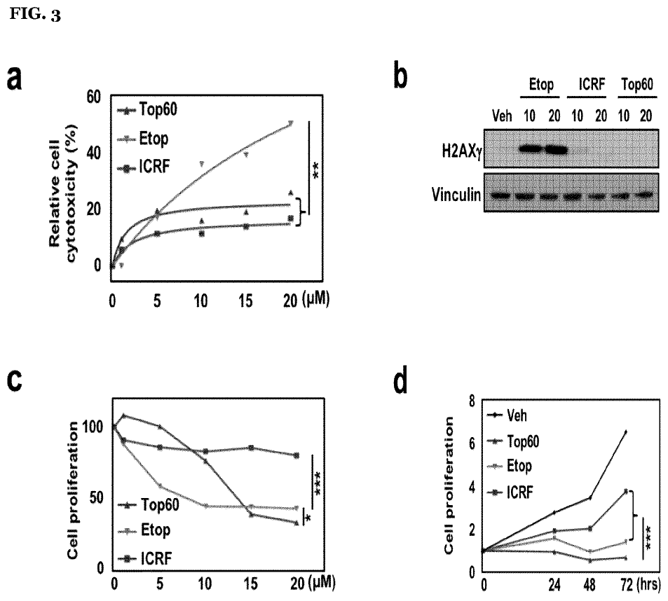

Second Round Screening With the discovered active compounds 19 and 23, a new screen was initiated to look for similar molecules. For this, the shape similarity screening program ROCS™¹⁰ was used The compounds chosen for the ROCS™ search was based on the docking conformation of Compound 23. The ZINC-15 library filtered down to 6 million molecules described previously was used for this screening. The top 62 molecules were hand-picked based on their ROCS™ similarity as well as their docking scores. These molecules were ordered and tested with the same medium throughput assays previously described. A large enrichment in the proportion of active molecules was observed, of close to 10-fold relative to the first round, validating the virtual screening pipeline. Finally, Compound 60 was discovered as the most active of the group, with a more potent inhibitory activity against Topo II than ICRF-193, and nanomolar IC₅₀ in both the decatenation and relaxation assays (see FIGS. 3a and 3b).

### Example 2: Characterization of Compound #60 (Top60)

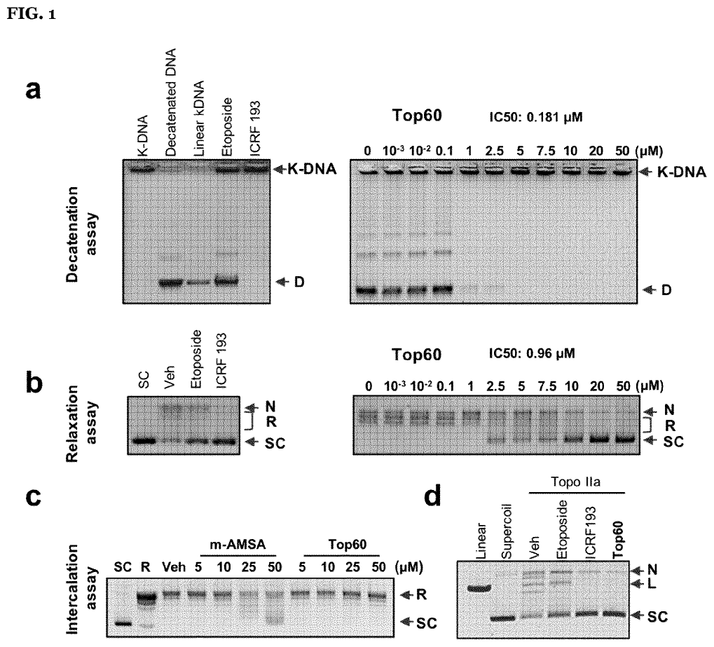

Top60 is a catalytic inhibitor of Topo II To verify that our lead Compound 60(Top60), the formation of ds DNA breaks was assayed both in and outside of cells. To assess whether Top60 causes double stranded DNA breaks outside cells, supercoiled plasmid was incubated with and without Top60 in the presence of Topo II (FIG. 1d). Satisfyingly, almost no band corresponding to linear DNA is formed and is similar in intensity to the known catalytic inhibitor ICRF-193. Furthermore, H2AXγ protein expression was assessed by Western blot to determine if the lead compound could cause DNA damage in cells, and compared to Etoposide as a control (FIG. 3b). Indeed, it was verified that Top60 does not cause DNA damage and is therefore in fact likely a catalytic inhibitor. Although Top60 may be a catalytic inhibitor, this does not preclude it from being an intercalator, which is unfavorable as intercalators may interfere with many processes which involve the manipulation of the DNA and cause off target affects. To determine if Top60 was capable of intercalating DNA, it was incubated with relaxed plasmid at increasing concentrations and compared with m-AMSA, a known intercalator. This can be visualized because intercalators will put negative supercoils in DNA, which will run more quickly on a gel than their relaxed counterparts. The results indicate that Top60 is not an intercalator (FIG. 1c).

### Example 3: Compound #60 (Top60) Causes Cancer Cell Growth to Stall

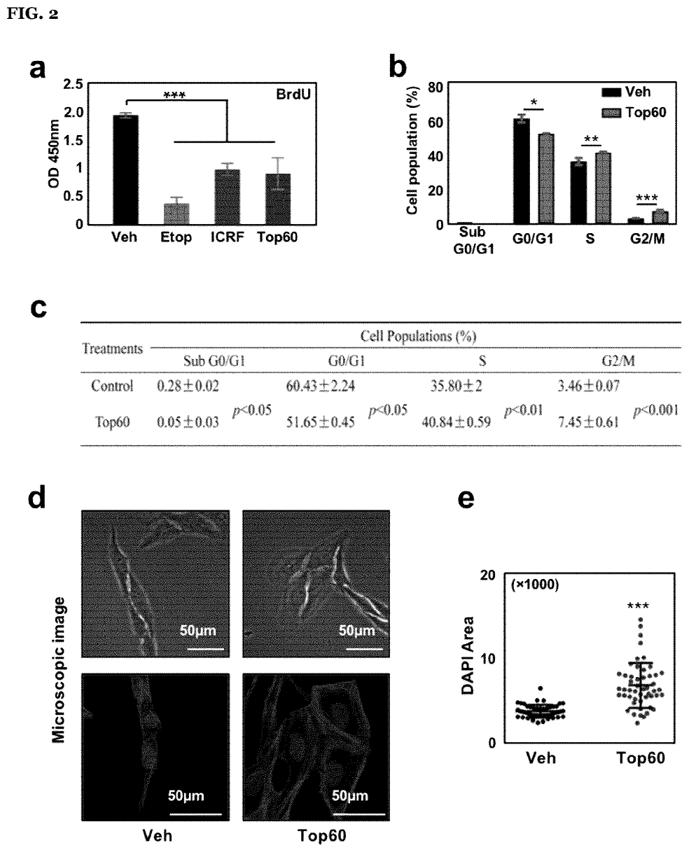

Decreased cellular replication was observed in Hela cells as evidenced by the BrdU assay (FIG. 2a). To further investigate the observed drop in replication rate, FACS was performed with PI staining (FIGS. 2b and 2c). As Topo II is important for making DNA accessible for replication, it is no surprise that there is a large cell population delayed in S-phase. Furthermore, the decatenation checkpoint is hypothesized to be in G2¹⁵, before M phase and is thus concordant with an increased cell population in G2/M (FIGS. 2b and 2c). To visualize the effect of stalled cell growth, fluorescence microscopy with DAPI staining was performed and cells treated with and without Top60. Indeed it is characteristic of catalytic inhibition to observe such enlarged nuclei as is seen in FIGS. 2d and 2e. This may happen when DNA is replicated, but not properly separated by the mitotic spindle, causing the nucleus to enlarge.

### Example 4: Top60 is a Non-Toxic Inhibitor of Topo II Dependent Cancer Cells

Low cytotoxic affect is observed from Top60 on Hela cells, comparable to ICRF (FIG. 3a). As catalytic inhibitors both ICRF and Top60 do not cause DNA damage as shown by H2AXγ protein levels. However, Top60 has strong inhibitory effects on k562 cell proliferation in both time- and dose-dependent experiments, with effects comparable to Etoposide (FIGS. 3c and 3d).

### Example 5: On-Target Effects of Top60 in Cells

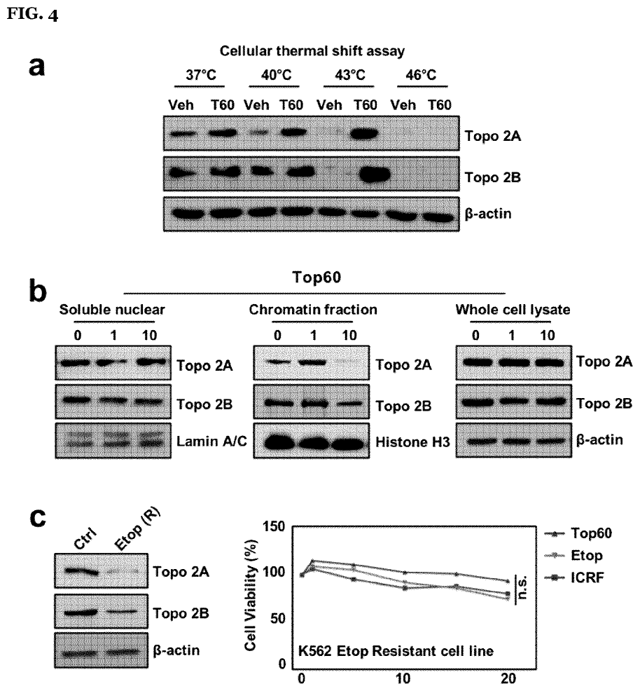

In order to asses binding of the lead compound to Topo II, a thermal shift assay was performed. This assay demonstrates binding by heat degradation of the protein, for example if a small molecule binds the protein, it will stabilize the protein against degradation at higher temperature than without. It was observed that Topo IIα was stabilized up to 43° C. in the presence of the lead compound (FIG. 4A). Furthermore, cellular fractionation was performed to assess localized changes in Topo II in response to compound treatment. As Topo IIα is important for decatenating DNA during cellular replication, it is expected to be chromatin bound. It was observed that upon treatment with Top60, Topo IIα became unbound from chromatin (and to a lesser extent Topo IIβ), while the total amount in the nucleus remained constant. To further verily that Topo II is the only observable target of Top60, Etoposide resistant cell lines were bred. These cells become resistant to Etoposide by downregulating Topo II proteins. The inhibitory effects of Top60 on these cells were dramatically reduced. These results support our hypothesis that the inhibitory effects of Top60 is mediated only through Topo II proteins in cells.

### Example 6: Molecular Origins of Binding Affinity

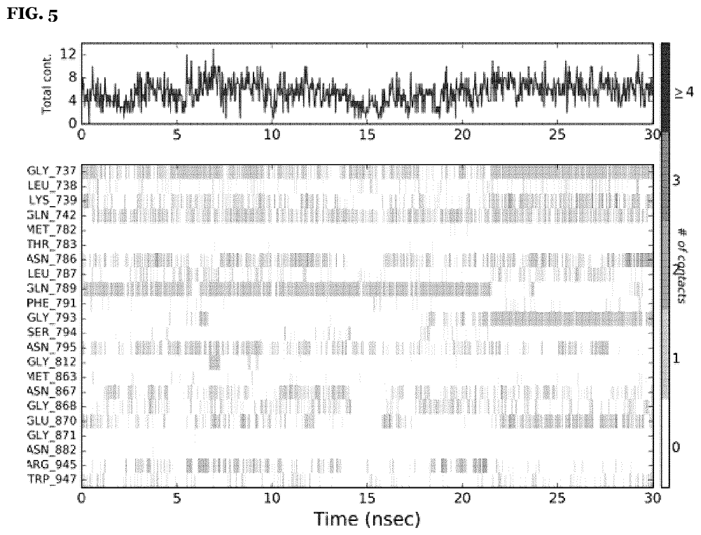

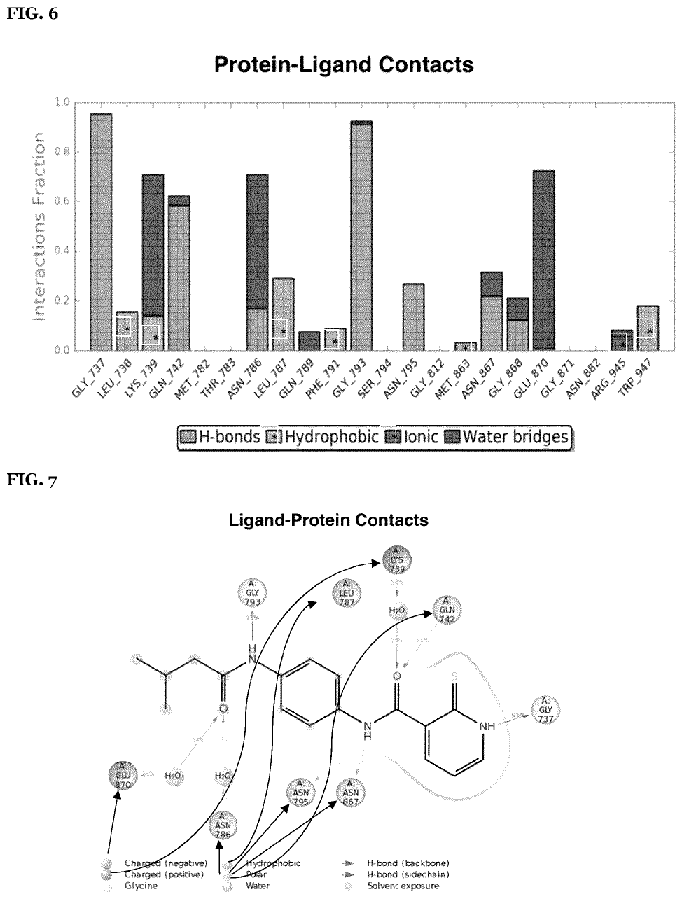

To understand the binding efficiency of the discovered small molecules, an MD simulation of compound 23 was performed for 30 nanoseconds. Throughout the simulation, H-bonding interactions are observed with G737, Q742, N786, Q789, G793, N795, N867, N868, and R945. With the strongest interactions in order being G737>G742>N795>Q789>G793>G868 (FIG. 5). Interestingly the Q789 interaction was found to be mutually exclusive with G793, this represents two alternative conformations observed throughout the simulation where the hydrogen bond donor amide para to the pyridinothione-amide of the ligand interact either with the sidechain carbonyl of Q789 or the backbone carbonyl of G793 after 22 nano-seconds (FIG. 5). The second conformation is believed to represent a stable conformation as the RMSD does not change much at this point. This observation prompted us to visualize and analyze the protein-ligand interactions after the 22 nano-second cut off (FIG. 6). The amide next to pyridinothione is stably interacting with the protein throughout the simulation, with the carbonyl hydrogen bonded to the backbone of G868, and with the NH of the ligand bonded through a very stable water bridge to N786 (FIG. 6). The strongest interactions are shown below (FIG. 7). In comparing the final snap-shot from the simulation with the docked structure, some induced fitting is observed where N786 which has swung over towards the ligand to form a hydrogen bonding interaction through a water bridge interaction with the carbonyl of the ligand amide. Overall the protein structure has deformed in a favourable way to the ligand as apparent by 3.38 Å Δ RMSD with the initial docked structure. The stable conformation observed later on in the simulation has about 1.5 Å Δ RMSD with the initial docked conformation. However, the first observed conformations is almost the same as the docked conformation, at 0.5 Å Δ RMSD where the variation is due to some minor readjusting in the pocket.

### Example 7: Testing of Alternative Compounds for Topoisomerase II Catalytic Inhibition

Numerous compounds were tested (except where indicated) as shown in TABLES 1 and 2 below, wherein active compounds or predicted active compounds (not tested) are shown in TABLE 1 and inactive compounds are shown in TABLE 2.

TABLE 1
FORMULA II AND III STRUCTURES TESTED WITH
TOPOISOMERASE II INHIBITORY ACTIVITY

| | | | | Cancer |
|---|---|---|---|---|
| | | | | Cell |
| Compound | | | TOP2 | Growth |
| # | Structure | Product ID | Inhib. | Inhib. |
| 23 | 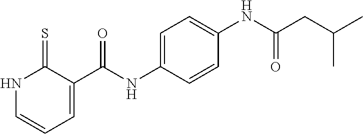 [SMILES: CC(C)CC(=O)Nc1ccc(NC(=O)c2ccc[nH]c2=S)cc1] | ZINC000026390134 | Y | Y |
| 60 | 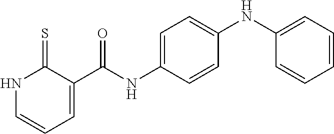 [SMILES: O=C(Nc1ccc(Nc2ccccc2)cc1)c1ccc[nH]c1=S] | ZINC29401178 | Y | Y |
| 62 | 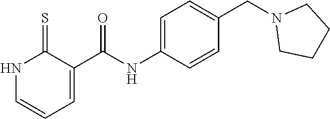 [SMILES: O=C(Nc1ccc(CN2CCCC2)cc1)c1ccc[nH]c1=S] | ZINC000030606160 | Y | Y |
| 63 | 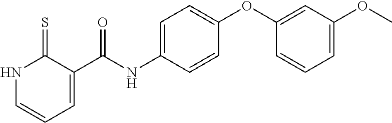 [SMILES: COc1cccc(Oc2ccc(NC(=O)c3ccc[nH]c3=S)cc2)c1] | ZINC000132036984 | Y | Y |
| 64 | 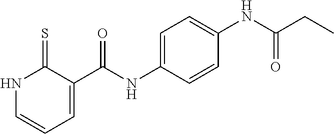 [SMILES: CCC(=O)Nc1ccc(NC(=O)c2ccc[nH]c2=S)cc1] | ZINC000053664991 | Y | Y |
| 65 | 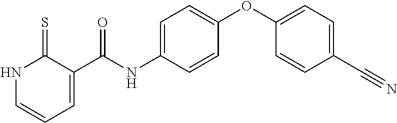 [SMILES: N#Cc1ccc(Oc2ccc(NC(=O)c3ccc[nH]c3=S)cc2)cc1] | ZINC000070840823 | Y | Y |
| 67 | 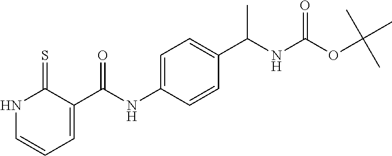 [SMILES: CC(NC(=O)OC(C)(C)C)c1ccc(NC(=O)c2ccc[nH]c2=S)cc1] | ZINC000173853553 | Y | Y |
| 68 | 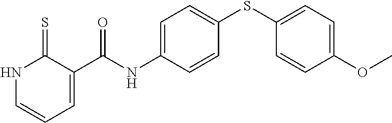 [SMILES: COc1ccc(Sc2ccc(NC(=O)c3ccc[nH]c3=S)cc2)cc1] | MCULE-3610729385 | Y | Y |
| 70 | 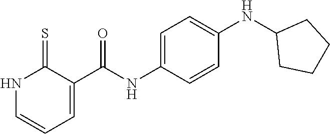 [SMILES: O=C(Nc1ccc(NC2CCCC2)cc1)c1ccc[nH]c1=S] | ZINC000109897726 | Y | Y |
| 72 | 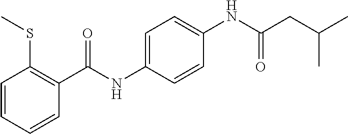 [SMILES: CSc1ccccc1C(=O)Nc1ccc(NC(=O)CC(C)C)cc1] | ZINC000025765627 | Y | Y |
| 75 | 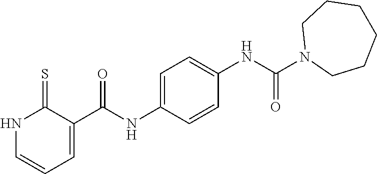 [SMILES: O=C(Nc1ccc(NC(=O)N2CCCCCC2)cc1)c1ccc[nH]c1=S] | ZINC000078501948 | Y | Y |
| 77 | 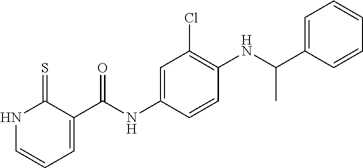 [SMILES: CC(Nc1ccc(NC(=O)c2ccc[nH]c2=S)cc1Cl)c1ccccc1] | ZINC000078505335 | Y | Y |
| 88 | 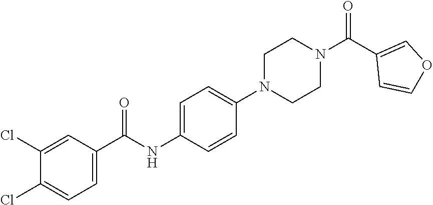 [SMILES: O=C(Nc1ccc(N2CCN(C(=O)c3ccoc3)CC2)cc1)c1ccc(Cl)c(Cl)c1] | ZINC000004109552 | Y | Y |
| 89 | 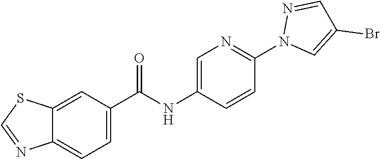 [SMILES: O=C(Nc1ccc(-n2cc(Br)cn2)nc1)c1ccc2ncsc2c1] | ZINC000072400849 | Y | Y |
| 90 | 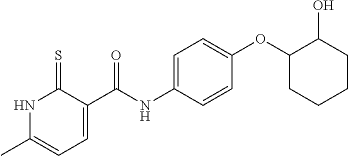 [SMILES: Cc1ccc(C(=O)Nc2ccc(OC3CCCCC3O)cc2)c(=S)[nH]1] | ZINC000118700593 | Y | Y |
| 110 | 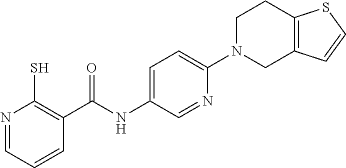 [SMILES: O=C(Nc1ccc(N2CCc3sccc3C2)nc1)c1cccnc1S] | ZINC000012851330 | Y | Y |
| 112 | 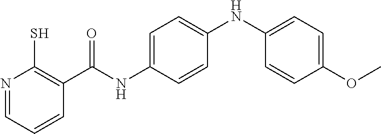 [SMILES: COc1ccc(Nc2ccc(NC(=O)c3cccnc3S)cc2)cc1] | ZINC40033795 | Y | Y |
| 113 | 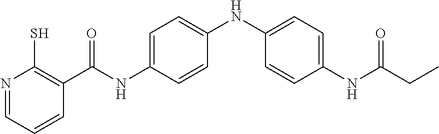 [SMILES: CCC(=O)Nc1ccc(Nc2ccc(NC(=O)c3cccnc3S)cc2)cc1] | ZINC000053664991 | Y | Y |
| 115 | 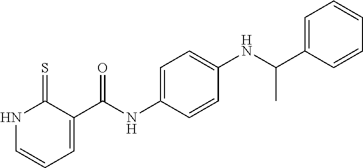 [SMILES: CC(Nc1ccc(NC(=O)c2ccc[nH]c2=S)cc1)c1ccccc1] | MCULE-1789094028 | Y | Y |
| Not assigned | 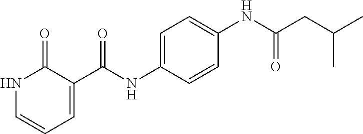 [SMILES: CC(C)CC(=O)Nc1ccc(NC(=O)c2ccc[nH]c2=O)cc1] | CAS# 10311197-27-9 | Not tested | Not tested |
| Not assigned | 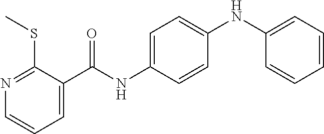 [SMILES: CSc1ncccc1C(=O)Nc1ccc(Nc2ccccc2)cc1] | CAS# 1323927-44-1 | Not tested | Not tested |
| Not assigned | 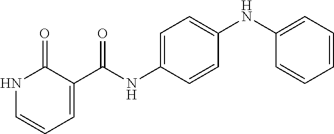 [SMILES: O=C(Nc1ccc(Nc2ccccc2)cc1)c1ccc[nH]c1=O] | CAS# 1286069-76-8 | Not tested | Not tested |
| 19 | 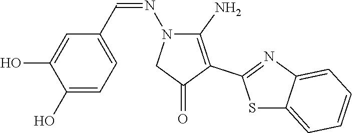 [SMILES: NC1=C(c2nc3ccccc3s2)C(=O)CN1/N=C\c1ccc(O)c(O)c1] | Enamine ID #Z55549767 | Y | Y |

Y is an indication that activity was found.?

TABLE 2
COMPOUNDS TESTED WITHOUT TOPOISOMERASE
II INHIBITORY ACTIVITY

| Compound # | Product ID |
|---|---|
| 0 | ZINC000012138105 |
| 1 | ZINC000253535516 |
| 2 | ZINC000056290401 |
| 3 | ZINC000033138821 |
| 4 | ZINC000065232688 |
| 5 | ZINC000010021911 |
| 6 | ZINC000076094337 |
| 7 | ZINC000097048428 |
| 8 | ZINC000043160373 |
| 9 | ZINC000069570110 |
| 10 | ZINC000072294999 |
| 11 | ZINC000089873057 |
| 12 | ZINC000097146884 |
| 13 | ZINC000058558309 |
| 14 | ZINC000027270515 |
| 15 | ZINC000029656248 |
| 16 | ZINC000031718062 |
| 17 | ZINC000048236816 |
| 18 | ZINC000007655156 |
| 20 | ZINC000007757740 |
| 21 | ZINC000014124930 |
| 22 | ZINC000017302992 |
| 24 | ZINC000029020806 |
| 25 | ZINC000004121086 |
| 26 | ZINC000005314802 |
| 27 | ZINC000070639923 |
| 28 | ZINC000004583665 |
| 29 | ZINC000022506496 |
| 30 | ZINC000217988681 |
| 31 | ZINC000005299934 |
| 32 | ZINC000049027723 |
| 33 | ZINC000218004659 |
| 34 | ZINC000013152939 |
| 35 | ZINC000000453760 |
| 36 | ZINC000000813387 |
| 37 | ZINC000004458432 |
| 38 | ZINC000025249817 |
| 39 | ZINC000041121250 |
| 40 | ZINC000072243188 |
| 41 | ZINC000000255265 |
| 42 | ZINC000004412820 |
| 43 | ZINC000000950359 |
| 44 | ZINC000000142256 |
| 45 | ZINC000000288074 |
| 46 | ZINC000004702221 |
| 47 | ZINC000002367467 |
| 48 | ZINC000072062671 |
| 49 | ZINC000009135106 |
| 50 | ZINC000218183463 |
| 51 | ZINC000001017748 |
| 52 | ZINC000023502408 |
| 54 | ZINC000009476592 |
| 55 | ZINC000013685141 |
| 56 | ZINC000013730275 |
| 57 | ZINC000013693334 |
| 58 | ZINC000009452637 |
| 59 | ZINC000003266646 |
| 61 | Enamine # Z27654153 |
| 66 | ZINC000126005270 |
| 69 | ZINC000029952395 |
| 71 | ZINC000054030958 |
| 73 | ZINC000171130539 |
| 74 | ZINC000071832549 |
| 76 | ZINC00001405245 |
| 78 | Enamine # Z27628972 |
| 79 | Enamine # Z357043432 |
| 80 | Enamine # Z454005294 |
| 81 | Enamine # Z1082379692 |
| 82 | Enamine # Z357042942 |
| 83 | Life Chemicals # F58340643 |
| 84 | Life Chemicals # F58336076 |
| 85 | ZINC000000724978 |
| 86 | ZINC000010109711 |
| 87 | ZINC000000804980 |
| 91 | Enamine # Z32139151 |
| 92 | ZINC000012948991 |
| 93 | ZINC000024706184 |
| 94 | ZINC000011363056 |
| 95 | ZINC000071909709 |
| 96 | ZINC000064663672 |
| 97 | ZINC000095443760 |
| 98 | ZINC000134663309 |
| 99 | ZINC000084358145 |
| 100 | ZINC000028835777 |
| 101 | ZINC000032889591 |
| 102 | Enamine # Z316519946 |
| 103 | ZINC000031718346 |
| 104 | ZINC000044917703 |
| 105 | Enamine # Z757862518 |
| 106 | ZINC000002632938 |
| 107 | Enamine # Z2241094364 |
| 108 | ZINC000012749182 |
| 109 | ZINC000012789151 |
| 111 | Enamine # Z367545462 |
| 114 | Enamine # Z224667642 |

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings.

### REFERENCES

1. Pommier, Y.; Leo, E.; Zhang, H.; Marchand, C., DNA topoisomerases and their poisoning by anticancer and antibacterial drugs. Chemistry & biology 2010, 17 (5), 421-33.
2. Felix, C. A.; Kolaris, C. P.; Osheroff, N., Topoisomerase II and the etiology of chromosomal translocations. DNA repair 2006, 5 (9-10), 1093-108.
3. Bailly, C., Contemporary challenges in the design of topoisomerase II inhibitors for cancer chemotherapy. Chemical reviews 2012, 112 (7), 3611-40.
4. Sterling, T.; Irwin, J. J., ZINC 15-Ligand Discovery for Everyone. J Chem Inf Model 2015, 55 (11), 2324-2337.
5. Axerio-Cilies, P.; Lack, N. A.; Nayana, M. R.; Chan, K. H.; Yeung, A.; Leblanc, E.; Guns, E. S.; Rennie, P. S.; Cherkasov, A., Inhibitors of androgen receptor activation function-2 (AF2) site identified through virtual screening. Journal of medicinal chemistry 2011, 54 (18), 6197-205.
6. Molecular Operating Environment (MOE), 2013.08; Chemical Computing Group ULC, 1010 Sherbooke St. West, Suite #910, Montreal, QC, Canada, H3A 2R7, 2018.
7. Friesner, R. A., Banks, J. L., Murphy, R. B., Halgren, T. A., Klicic, J. J., Mainz, D. T., Repasky, M. P., Knoll, E. H., Shelley, M., Perry, J. K., et al. (2004). Glide: a new approach for rapid, accurate docking and scoring. 1. Method and assessment of docking accuracy. J. Med. Chem. 47, 1739-1749.8. Zsoldos, Z., Reid, D., Simon, A., Sadjad, S. B., and Johnson, A. P. (2007).
8. eHiTS: a new fast, exhaustive flexible ligand docking system. J. Mol. Graph. Model. 26, 198-212.
9. Li, X. F.; Deshmukh, R.; Shebley, M.; de Morais, S. M., Evaluation of AD MET properties predictions using the advanced in silico modeling tool ADMET predictor. Drug Metab Rev 2011, 43, 133-134.
10. Hawkins, P. C. D.; Skillman, A. G. and Nicholls, A., Comparison of Shape-Matching and Docking as Virtual Screening Tools, Journal of Medicinal Chemistry, Vol. 50, pp. 74-82, 2007.
11. Borrel, A.; Regad, L.; Xhaard, H.; Petitjean, M.; Camproux, A. C., Pock Drug: A Model for Predicting Pocket Draggability That Overcomes Pocket Estimation Uncertainties. J Chem Inf Model 2015, 55 (4), 882-895.
12. D. A. Case, I. Y. Ben-Shalom, S. R. Brozell, D. S. Cerutti, T. E. Cheatham, III, V. W. D. Cruzeiro, T. A. Darden, R. E. Duke, D. Ghoreishi, M. K. Gilson, H. Gohlke, A. W. Goetz, D. Greene, R Harris, N. Homeyer, S. Izadi, A. Kovalenko, T. Kurtzman, T. S. Lee, S. LeGrand, P. Li, C. Lin, J. Liu, T. Luchko, R. Luo, D. J. Mermelstein, K. M. Merz, Y. Miao, G. Monard, C. Nguyen, H. Nguyen, I. Omelyan, A. Onufriev, F. Pan, R. Qi, D. R. Roe, A. Roitberg, C. Sagui, S. Schott-Verdugo, J. Shen, C. L. Simmerling, J. Smith, R. Salomon-Ferrer, J. Swails, R. C. Walker, J. Wang, H. Wei, R. M. Wolf, X. Wu, L. Xiao, D. M. York and P. A. Kollman (2018), AMBER 2018, University of California, San Francisco.
13. Katritzky, A. R.; Jug, K.; Oniciu, D. C., Quantitative measures of aromaticity for mono-, bi-, and tricyclic penta- and hexaatomic heteroaromatic ring systems and their interrelationships. Chem Rev 2001, 101 (5), 1421-1449.
14. Forlani, L.; Cristoni, G.; Boga, C.; Todesco, P. E.; Del Vecchio, E.; Selva, S.; Monari, M., Reinvestigation of the tautomerism of some substituted 2-hydroxypyridines. Arkivoc 2002, 198-215.
15. Downes C S, Clarke D J, Mullinger A M, Gimenez-Abian J F, Creighton A M, Johnson R T (1994) A topoisomerase II-dependent G2 cycle checkpoint in mammalian cells. Nature 372: 467-470
16. A. R. Katritzky, K. Jug, and D. C. Oniciu. Quantitative measures of aromaticity for mono-, bi-, and tricyclic penta- and hexaatomic heteroaromatic ring systems and their interrelationships. Chemical reviews, 101(5):1421-1450, 2001.
17. L. Forlani, G. Cristoni, C. Boga, P. E. Todesco, E. Del Vecchio, S. Selva, and M. Monari. Reinvestigation of the tautomerism of some substituted 2-hydroxypyridines. Arkivoc, 11:198-215, 2002.
18. Wysocka J, Reilly P T, Herr W. Loss of HCF-1-chromatin association precedes temperature-induced growth arrest of tsBN67 cells. Mol Cell Biol. 21(11):3820-9, 2001.
19. Li H, Xie N, Chen R, Verreault M, Fazli L, Gleave M E, Barbier O, Dong X UGT2B17 Expedites Progression of Castration-Resistant Prostate Cancers by Promoting Ligand-Independent AR Signaling. Cancer Res.; 76(22):6701-6711, 2016. doi: 10.1158/0008-5472.CAN-16-1518. Epub 2016 Sep. 22.
20. McClendon A K, Rodriguez A C, Osheroff N. Human topoisomerase IIalpha rapidly relaxes positively supercoiled DNA: implications for enzyme action ahead of replication forks. J Biol Chem.; 280(47):39337-45, 2005.

1. A method of inhibiting Topoisomerase II, the method comprising administering a compound having the structure of Formula II: 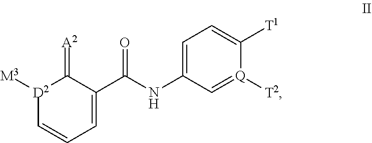
wherein,
A² is selected from S and O;
D² is selected from N and CH;
M³ is H;
Q is selected from C and N;
T¹ is selected from H, 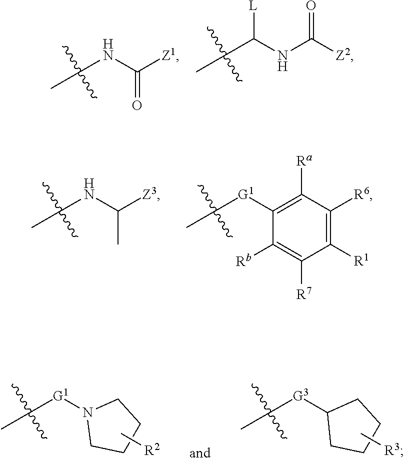
T² is absent when Q is N or is selected from H, F, Cl, Br, 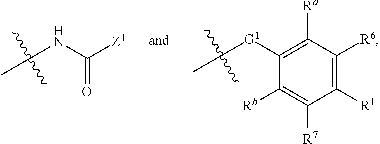
when Q is C, provided that both T¹ and T² are not both H;
Z¹ is selected from H, CH₂CH₃, CH₂CH₂CH₃, CH(CH₃)CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, OC(CH₃) (CH₃)CH₃, 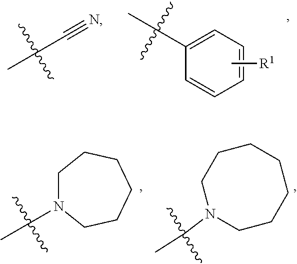
F, Cl, Br and CF₃;
Z² is selected from H, CH₃, CH₂CH₃, CH₂CH₂CH₃, CH(CH₃)CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, OC(CH₃) (CH₃)CH₃, 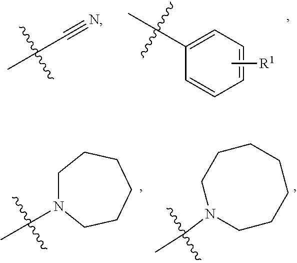
F, Cl, Br and CF₃;
Z³ is selected from H, CH₃, CH₂CH₃, CH₂CH₂CH₃, CH(CH₃)CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, OC(CH₃) (CH₃)CH₃, 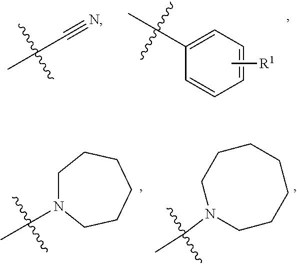
F, Cl, Br and CF₃;
L is selected from H, CH₃, CH₂CH₃, CH(CH₃)CH₃, NH₂, OH, OCH₃, OCH₂CH₃, F, Cl, Br and CF₃;
G¹ is selected from O, S, CH₂ and NH;
G² is selected from O, S, CH₂ and NH;
G³ is selected from O, S, CH₂ and NH;
R^{a} and R^{b} are H;
R¹ is independently selected from H, CH₃, CH₂CH₃, CH₂CH₂CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, NHC(═O)CH₂CH₃, 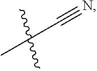 [SMILES: *C#N]
F, Cl, Br and CF₃;
R² is independently selected from H, CH₃, CH₂CH₃, CH₂CH₂CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, NHC(═O)CH₂CH₃, 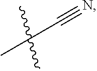 [SMILES: *C#N]
F, Cl, Br and CF₃;
R³ is independently selected from H, CH₃, CH₂CH₃, CH₂CH₂CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, NHC(═O)CH₂CH₃, 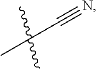 [SMILES: *C#N]
F, Cl, Br and CF₃;
R⁶ is selected from H, CH₃, CH₂CH₃, CH₂CH₂CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, NHC(═O)CH₂CH₃, 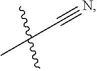 [SMILES: *C#N]
F, Cl, Br and CF₃; and
R⁷ is selected from H, CH₃, CH₂CH₃, CH₂CH₂CH₃, CH₂CH(CH₃)CH₃, CH(CH₃)CH₃, OCH₃, OCH₂CH₃, OCH₂CH₂CH₃, OCH₂CH(CH₃)CH_{3J} OCH(CH₃)CH₃, NHC(═O)CH₂CH₃, 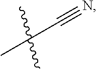 [SMILES: *C#N]
F, Cl, Br and CF₃.

2. A method of inhibiting Topoisomerase II, the method comprising administering a compound having the structure of Formula III: 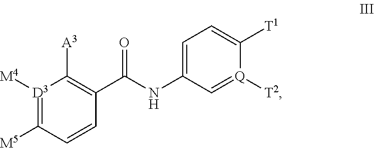
wherein,
A³ is selected from SH, OH and OCH₃;
D³ is selected from N and C;
alternatively, As is H or SCH₃ when D³ is C;
M⁴ is absent or is selected from H, Cl, F, Br;
M⁵ is selected from H, CH₃, Cl, F and Br;
Q is selected from C and N;
T¹ is selected from H, 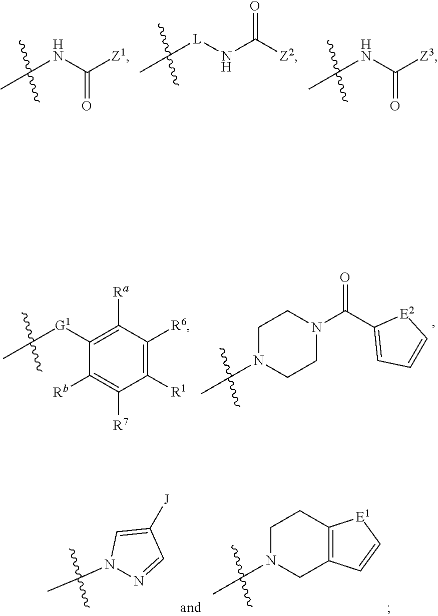
T² is absent when Q is N or is selected from H, F, Cl, Br, 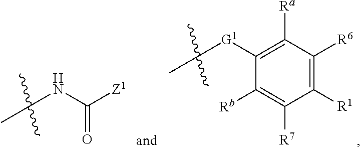
when Q is C, provided that both T¹ and T² are not both H;
Z¹ is selected from H, CH₂CH₃, CH₂CH₂CH₃, CH(CH₃)CH₃, CH₂CH(CH₃)CH₃, CH(CH₃)CH₃, OCH₃, OCH₂CH₃, OCH₂CH₂CH₃, OCH₂CH(CH₃)CH_{3J} OCH(CH₃)CH₃, OC(CH₃) (CH₃)CH₃, 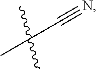 [SMILES: *C#N]
F, Cl, Br and CF₃;
Z² is selected from H, CH₃, CH₂CH₃, CH₂CH₂CH₃, CH(CH₃)CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, OC(CH₃) (CH₃)CH₃, 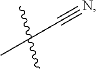 [SMILES: *C#N]
F, Cl, Br and CF₃;
Z³ is selected from H, CH₃, CH₂CH₃, CH₂CH₂CH₃, CH(CH₃)CH₃, CH₂CH(CH₃)CH₃, CH(CH₃)CH₃, OCH₃, OCH₂CH₃, OCH₂CH₂CH₃, OCH₂CH(CH₃)CH₃, OCH(CH₃)CH₃, OC(CH₃) (CH₃)CH₃, 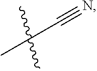 [SMILES: *C#N]
F, Cl, Br and CF₃;
L is selected from H, CH₃, CH₂CH₃, CH(CH₃)CH₃, NH₂, OH, OCH₃, OCH₂CH₃, F, Cl, Br and CF₃;
G¹ is selected from O, S, CH₂ and NH;
R^{a} and R^{b} are H;
R¹ is selected from OCH₃ and NHC(═O)CH₂CH₃;
R⁶ is selected from H, CH₂CH₂CH₃, CH₂CH(CH₃)CH₃, CH(CH₃)CH₃, OCH₂CH₃, OCH₂CH₂CH₃, OCH₂CH(CH₃)CH₃, OCH(CH₃)CH₃, NHC(═O)CH₂CH₃, 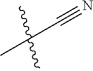 [SMILES: *C#N]
and CF₃;
R⁷ is selected from H, CH₂CH₂CH₃, CH₂CH(CH₃)CH₃, CH(CH₃)CH₃, OCH₂CH₃, OCH₂CH₂CH₃, OCH₂CH(CH₃)CH₃, OCH(CH₃)CH₃, NHC(═O)CH₂CH₃, 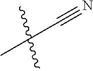 [SMILES: *C#N]
and CF₃;
J is selected from H, CH₃, NH₂, OH, OCH₃, F, Cl, Br and CF₃;
E¹ is selected from O, S, CH₂ and NH; and
E² is selected from O, S, CH₂ and NH.

3. The method of claim 1 or 2, wherein the inhibiting Topoisomerase II is catalytic inhibition.

4. The method of any one of claims 1-3, wherein the inhibiting Topoisomerase II is for the treatment of cancer.

5. The method of claim 4, wherein the cancer is selected from one or more of the following: cervical cancer; small-cell lung cancer; testicular cancer; carcinoma; neuroblastoma; osteosarcoma; glioblastoma; melanoma; lymphoma; leukemia; esophageal cancer; stomach cancer; colon cancer; breast cancer; ovarian cancer; endometrial cancer; chondrosarcomas; central nervous system cancer; liver cancer; and prostate cancer.

6. The method of claim 5, wherein the prostate cancer is selected from: Neuroendocrine Prostate Cancer (NEPC); Prostate Adenocarcinoma; castration resistant prostate cancer (CRPC); androgen receptor pathway inhibitor (ARPI) resistant prostate cancer; enzalutamide (ENZ)-resistant (ENZ^{R}); and Abiraterone (Abi)-resistant (ABI_{R}).

7. The method of any one of claims 1-6, wherein the compound is selected from one or more of the following:

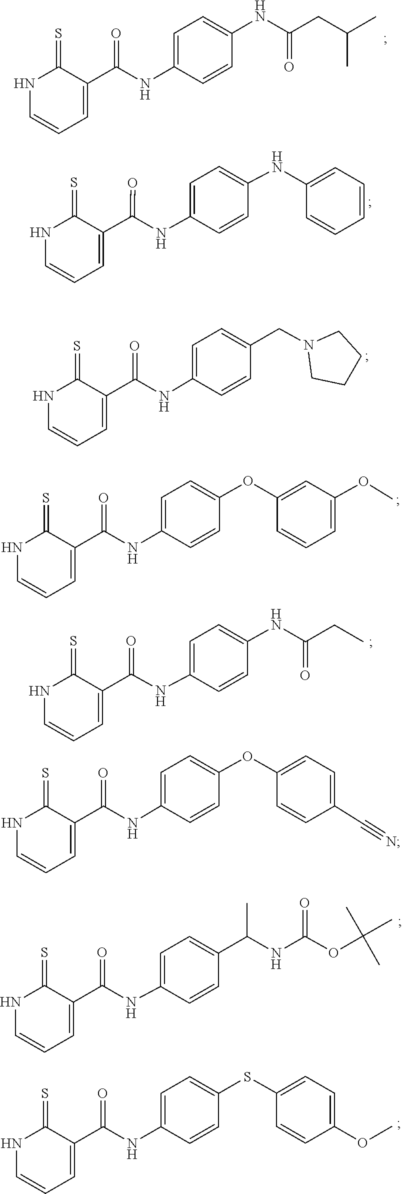

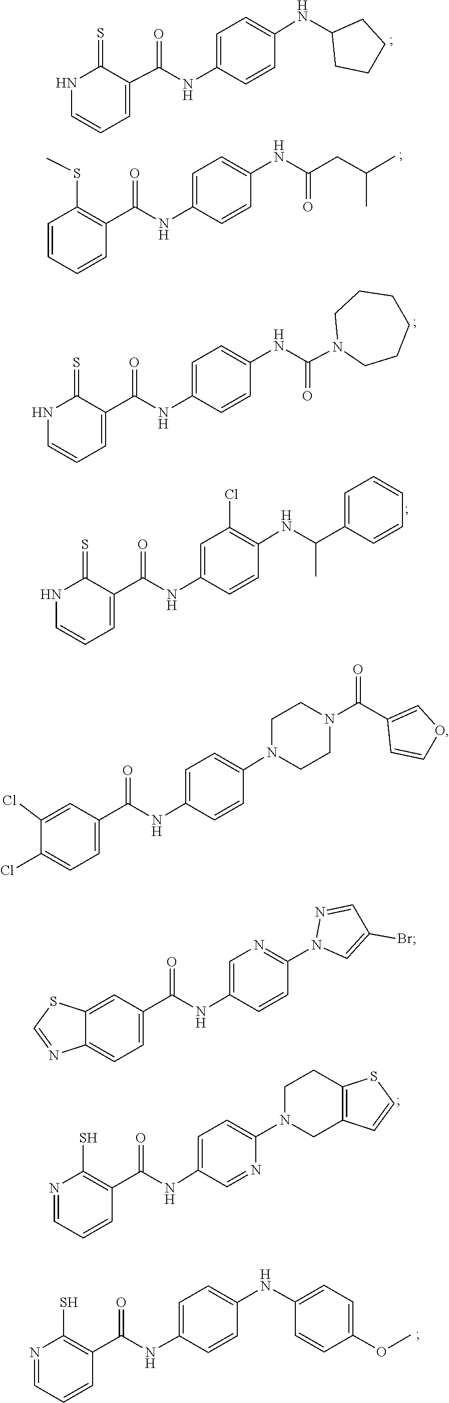

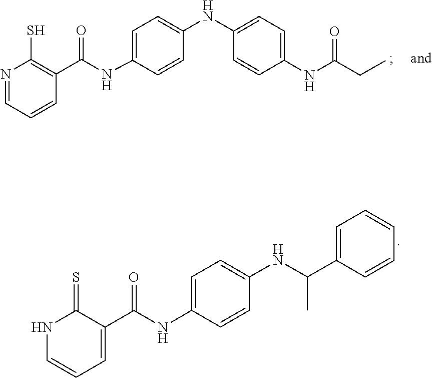 [SMILES: CCC(=O)Nc1ccc(Nc2ccc(NC(=O)c3cccnc3S)cc2)cc1 ; CC(Nc1ccc(NC(=O)c2ccc[nH]c2=S)cc1)c1ccccc1]

8. The method of any one of claims 1-7, wherein the compound of Formula II or III is administered in combination with a taxol and/or a Topoisomerase poison and/or an androgen receptor (AR) therapy for the treatment of prostate cancer.

9. A pharmaceutical composition for treating cancer, comprising compound of Formula II or III and a pharmaceutically acceptable carrier.

10. The pharmaceutical composition of claim 9, wherein the cancer is selected from one or more of the following: cervical cancer; small-cell lung cancer; testicular cancer; carcinoma; neuroblastoma; osteosarcoma; glioblastoma; melanoma; lymphoma; leukemia; esophageal cancer; stomach cancer; colon cancer; breast cancer; ovarian cancer; endometrial cancer; chondrosarcomas; central nervous system cancer; liver cancer; and prostate cancer.

11. Use of a compound of Formula II or III for treating cancer.

12. Use of a compound of Formula II or III in the manufacture of a medicament for treating cancer.

13. The use of claim 11 or 12, wherein the cancer is selected from one or more of the following: cervical cancer; small-cell lung cancer; testicular cancer; carcinoma; neuroblastoma; osteosarcoma; glioblastoma; melanoma; lymphoma; leukemia; esophageal cancer; stomach cancer; colon cancer; breast cancer; ovarian cancer; endometrial cancer; chondrosarcomas; central nervous system cancer; liver cancer; and prostate cancer.

14. A commercial package comprising (a) a compound of any one of Formula II or III and a pharmaceutically acceptable carrier; and (b) instructions for the use thereof for treating cancer.

15. A commercial package comprising (a) a pharmaceutical composition comprising a compound of any one of Formula II or III and a pharmaceutically acceptable carrier; and (b) instructions for the use thereof for treating cancer.

16. The commercial package of claim 14 or 15, wherein the cancer is selected from one or more of the following: cervical cancer; small-cell lung cancer; testicular cancer; carcinoma; neuroblastoma; osteosarcoma; glioblastoma; melanoma; lymphoma; leukemia; esophageal cancer; stomach cancer; colon cancer; breast cancer; ovarian cancer; endometrial cancer; chondrosarcomas; central nervous system cancer; liver cancer; and prostate cancer.

17. A method of inhibiting Topoisomerase II, the method comprising administering a compound having the structure

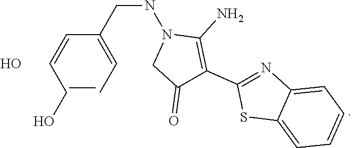

18. The method of claim 17, wherein the inhibiting Topoisomerase II is catalytic inhibition.

19. The method of claim 17 or 18, wherein the inhibiting Topoisomerase II is for the treatment of cancer.

20. The method of claim 19, wherein the cancer is selected from one or more of the following: cervical cancer; small-cell lung cancer; testicular cancer; carcinoma; neuroblastoma; osteosarcoma; glioblastoma; melanoma; lymphoma; leukemia; esophageal cancer; stomach cancer; colon cancer; breast cancer; ovarian cancer; endometrial cancer; chondrosarcomas; central nervous system cancer; liver cancer; and prostate cancer.

21. The method of claim 20, wherein the prostate cancer is selected from: Neuroendocrine Prostate Cancer (NEPC); Prostate Adenocarcinoma; castration resistant prostate cancer (CRPC); androgen receptor pathway inhibitor (ARPI) resistant prostate cancer; enzalutamide (ENZ)-resistant (ENZ^{R}); and Abiraterone (Abi)-resistant (ABI_{R}).

22. The method of any one of claims 17-21, wherein the compound is administered in combination with a taxol and/or a Topoisomerase poison and/or an androgen receptor (AR) therapy for the treatment of prostate cancer.

23. A compound for use in the treatment of cancer, the compound is selected from one or more of the following:

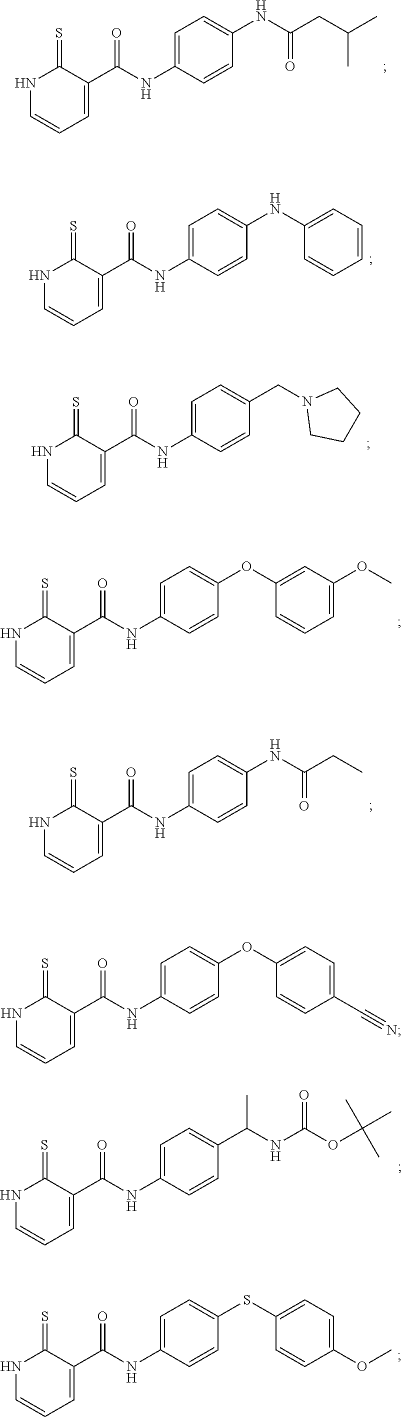

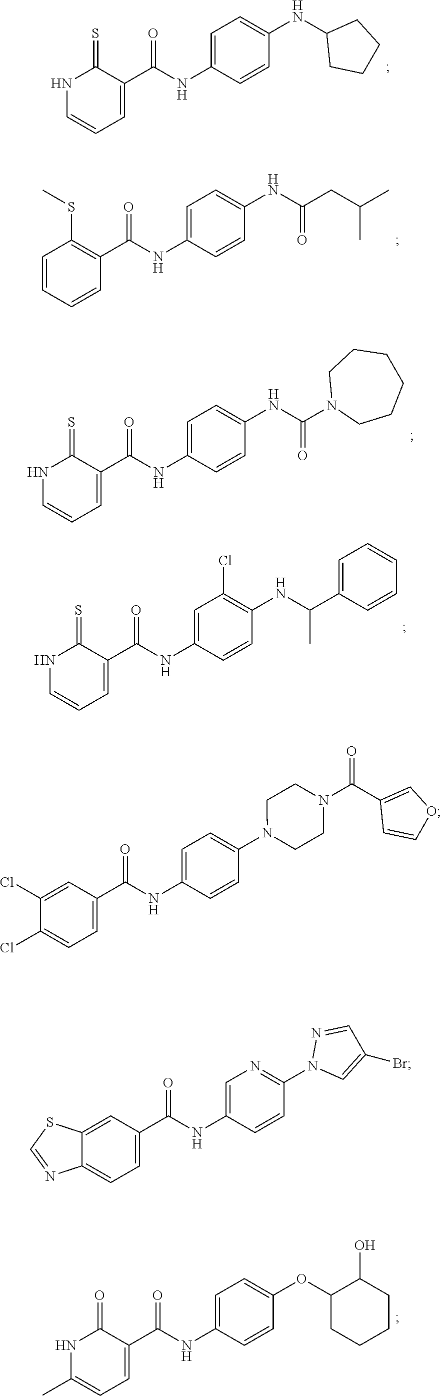

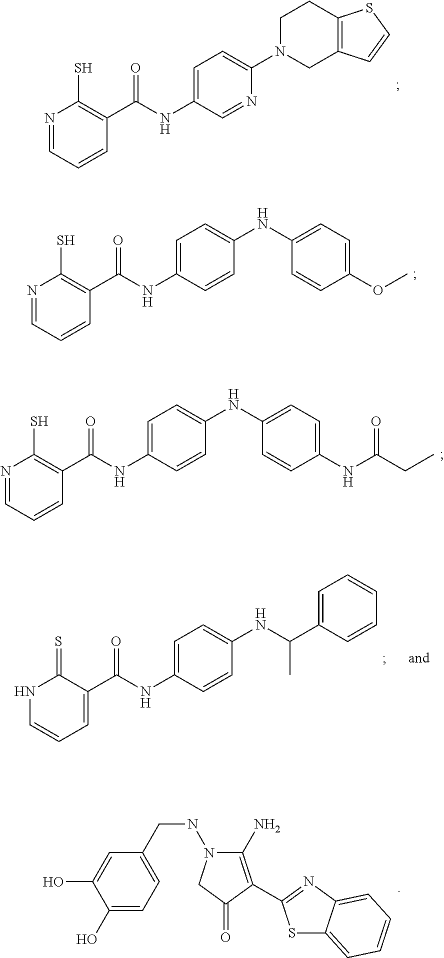 [SMILES: O=C(Nc1ccc(N2CCc3sccc3C2)nc1)c1cccnc1S ; COc1ccc(Nc2ccc(NC(=O)c3cccnc3S)cc2)cc1 ; CCC(=O)Nc1ccc(Nc2ccc(NC(=O)c3cccnc3S)cc2)cc1 ; CC(Nc1ccc(NC(=O)c2ccc[nH]c2=S)cc1)c1ccccc1 ; NC1=C(c2nc3ccccc3s2)C(=O)CN1NCc1ccc(O)c(O)c1]

24. The compound of claim 23, wherein the treatment of cancer is by inhibition of Topoisomerase II.

25. The compound of claim 23 or 24, wherein the treatment of cancer is by catalytic inhibition of Topoisomerase II.

26. The compound of claim 23, 24 or 25, wherein the cancer is selected from one or more of the following: cervical cancer; small-cell lung cancer; testicular cancer; carcinoma; neuroblastoma; osteosarcoma; glioblastoma; melanoma; lymphoma; leukemia; esophageal cancer; stomach cancer; colon cancer; breast cancer; ovarian cancer; endometrial cancer; chondrosarcomas; central nervous system cancer; liver cancer; and prostate cancer.

27. The compound of claim 26, wherein the prostate cancer is selected from: Neuroendocrine Prostate Cancer (NEPC); Prostate Adenocarcinoma; castration resistant prostate cancer (CRPC); androgen receptor pathway inhibitor (ARPI) resistant prostate cancer; enzalutamide (ENZ)-resistant (ENZ^{R}); and Abiraterone (Abi)-resistant (ABI_{R}).

28. The compound of any one of claims 23-27, wherein the compound is used in combination with a taxol and/or a Topoisomerase poison and/or an androgen receptor (AR) therapy for the treatment of prostate cancer.

29. The compound of any one of claims 23-28, wherein the compound is selected from 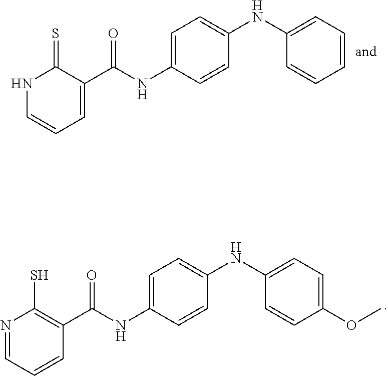 [SMILES: O=C(Nc1ccc(Nc2ccccc2)cc1)c1ccc[nH]c1=S ; COc1ccc(Nc2ccc(NC(=O)c3cccnc3S)cc2)cc1]
